(19) United States
(12) Patent Application Publication
(10) Pub. No.: US 2024/0269322 A1
(43) Pub. Date: Aug. 15, 2024

(54) ADENO-ASSOCIATED VIRUS VIRIONS AND METHODS OF USE THEREOF

(71) Applicants: Chan Zuckerberg Biohub, Inc., San Francisco, CA (US); The Regents of the University of California, Oakland, CA (US)

(72) Inventors: David V. Schaffer, Berkeley, CA (US); Danqing Zhu, Berkeley, CA (US); Tomasz J. Nowakowski, San Francisco, CA (US); Galina Schmunk, San Francisco, CA (US)

(21) Appl. No.: 18/031,294

(22) PCT Filed: Oct. 20, 2021
(86) PCT No.: PCT/US2021/055806

§ 371 Date: Apr. 11, 2023

Related U.S. Application Data
(60) Provisional application No. 63/094,820, filed on Oct. 21, 2020.

(51) Int. Cl. A61K 48/00; C07K 14/005; C12N 9/22; C12N 15/11; C12N 15/86

(52) U.S. Cl. CPC A61K 48/0041; C07K 14/005; C12N 9/22; C12N 15/11; C12N 15/86; C12N 2310/20; C12N 2750/14122; C12N 2750/14143

(57) ABSTRACT

The present disclosure provides recombinant adeno-associated virus (rAAV) virions comprising a variant AAV capsid protein. An rAAV virion of the present disclosure can exhibit greater infectivity of a macrophage. The present disclosure also provides methods of delivering a gene product to a target macrophage in an individual by administering to the individual an rAAV of the present disclosure. The present disclosure also provides treatment methods comprising administering to an individual in need thereof an rAAV virion of the present disclosure.

### CROSS-REFERENCE

This application claims the benefit of U.S. Provisional Patent Application No. 63/094,820, filed Oct. 21, 2020, which application is incorporated herein by reference in its entirety.

### INCORPORATION BY REFERENCE OF SEQUENCE LISTING PROVIDED AS A TEXT FILE

A Sequence Listing is provided herewith as a text file, “CZBH-001WO_SEQ_LIST_ST25” created on Sep. 28, 2021 and having a size of 371 KB. The contents of the text file are incorporated by reference herein in their entirety.

### INTRODUCTION

Adeno-associated virus (AAV) belongs to the Parvoviridae family and Dependovirus genus, whose members replicate upon co-infection with a helper virus such as adenovirus. AAV can establish a latent infection in the absence of a helper. Virions are composed of a 25 nm icosahedral capsid encompassing a 4.9 kb single-stranded DNA genome with two open reading frames: rep and cap. The non-structural rep gene encodes four regulatory proteins essential for viral replication, whereas cap encodes three structural proteins (VP1-3) that assemble into a 60-mer capsid shell. This viral capsid mediates the ability of AAV vectors to overcome many of the biological barriers of viral transduction-including cell surface receptor binding, endocytosis, intracellular trafficking, and unpackaging in the nucleus.

### SUMMARY

The present disclosure provides recombinant adeno-associated virus (rAAV) virions comprising a variant AAV capsid protein. An rAAV virion of the present disclosure can exhibit greater infectivity of a macrophage. The present disclosure also provides methods of delivering a gene product to a target macrophage in an individual by administering to the individual an rAAV of the present disclosure. The present disclosure also provides treatment methods comprising administering to an individual in need thereof an rAAV virion of the present disclosure.

### BRIEF DESCRIPTION OF THE DRAWINGS

FIG. 1 is a schematic depiction of a directed evolution workflow for AAV vector engineering targeting microglia cells.

FIG. 2 depicts data showing that natural AAV serotypes transduce human microglia cells in brain slices very poorly, exhibiting overall <5% of efficiency.

FIG. 3 depicts quantification of improved infectivity and specificity targeting microglia cells using an evolved AAV vector.

FIG. 4 provides representative images of AAV-GFP transduction towards microglia cells using evolved AAV clone versus. natural serotype.

FIG. 5 provides an amino acid sequence alignment of a variant AAV capsid (referred to as “FIG. 6B” in FIG. 5), and AAV capsids of AAV1, AAV2, AAV3A, AAV3B, AAV4, AAV5, AAV6, AAV7, AAV8, and AAV9 (from top to bottom SEQ ID NOs:53-63).

FIG. 6A-6B provide the amino acid sequence of exemplary AAV variants of the present disclosure (from top to bottom SEQ ID NOs:64 and 53).

FIG. 7A-7J provide amino acid sequences of AAV capsids of AAV1, AAV2, AAV3A, AAV3B, AAV4, AAV5, AAV6, AAV7, AAV8, and AAV9, respectively (from top to bottom SEQ ID NOs:54-63).

FIG. 8A-8P provide amino acid sequences of various CRISPR/Cas effector polypeptides.

FIG. 9A-9M provide amino acid sequences of exemplary AAV variants of the present disclosure (from top to bottom SEQ ID NOs: 81-93).

FIG. 10 depicts the calculated percent of amino acids at each of 32 variable positions in an AAV capsid protein. FIG. 10 shows the dominant amino acid frequency at 32 variable positions after three rounds of selection. If selection resulted in a change in amino acid identity at that position, the new amino acid and frequency is shown.

### DEFINITIONS

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the present disclosure described herein that is a polynucleotide may encompass both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wisconsin, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00;
Gap Penalty: 1.00;
Gap Size Penalty: 0.33; and
Joining Penalty: 30.0.

A “gene” refers to a polynucleotide containing at least one open reading frame that is capable of encoding a particular protein after being transcribed and translated.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein.

The term “antibody” includes nanobodies. The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

The term “antibody” includes single-chain Fv (scFv). “Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

A “small interfering” or “short interfering RNA” or siRNA is an RNA duplex of nucleotides that is targeted to a gene interest (a “target gene”). An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA (or “miRNA”) sequences have been described in publications such as Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179. Examples of microRNAs include any RNA that is a fragment of a larger RNA or is a miRNA, siRNA, stRNA, sncRNA, tncRNA, snoRNA, smRNA, shRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA that has been cleaved from a microRNA precursor (a “pre-miRNA”), or that has been synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides. neuroprotective polypeptides, and the like, when discussed in the context of delivering a gene product to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a gene product to a mammalian subject (which may be referred to as “transgenes” to be delivered to a recipient cell), include polynucleotides encoding the intact polypeptide or any fragment or genetically engineered derivative possessing the desired biochemical function.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

“AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), avian AAV, bovine AAV, canine AAV, equine AAV. primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” or “rAAV virion” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV virion”. Thus, production of rAAV virion necessarily includes production of an rAAV vector, as such a vector is contained within an rAAV virion.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA).

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments. rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

A “library” of rAAV virions is a composition containing a plurality of rAAV virions representing two or more varieties of rAAV virions that differ among each other in structure (e.g., structure of the AAV capsid protein) and/or sequence of the nucleic acids contained therein.

The term “tropism” refers to a viral particle having higher infectivity for one cell type compared to one or more other cell types. Tropism may also refer to the tissue specificity of the viral particle. For instance, a viral particle that has tropism for macrophage cells has a higher infectivity for macrophage cells compared to the infectivity for non-macrophage cells. For instance, a viral particle that has tropism for microglial cells has a higher infectivity for microglial cells compared to the infectivity for non-microglial cells. In AAV, tropism is affected by the AAV capsid serotype, i.e., the AAV capsid protein amino acid sequence. In contrast, a viral particle is said to be promiscuous when the viral particle exhibits infectivity for a broad range of cell types. In some cases, a viral particle exhibits tropism for one or more cell types, and may also be promiscuous.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Generally, the genetic element is introduced into a chromosome or mini-chromosome in the cell; but any alteration that changes the phenotype and/or genotype of the cell and its progeny is included in this term.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

The term “genome editing” refers to a process by which a genetic sequence within a cell is altered by inserting, replacing or removing sequences using heterologous nucleases. The heterologous nuclease may be a genetically engineered nuclease, including members of zinc finger nucleases, transcription activator-like effector nucleases (TALENs), Cas9/guide RNA (gRNA) system, or engineered meganucleases.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an rAAV virion” includes a plurality of such virions and reference to “the heterologous gene product” includes reference to one or more heterologous gene products and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides recombinant adeno-associated virus (rAAV) virions comprising a variant AAV capsid protein. An rAAV virion of the present disclosure can exhibit greater infectivity of a macrophage. The present disclosure also provides methods of delivering a gene product to a target macrophage in an individual by administering to the individual an rAAV of the present disclosure. The present disclosure also provides treatment methods comprising administering to an individual in need thereof an rAAV virion of the present disclosure.

### Recombinant AAV Virions With Variant Capsids

The present disclosure provides an rAAV virion that comprises a variant capsid protein, which variant capsid protein confers on the rAAV virion an increased ability to infect macrophages, compared to a control AAV virion comprising a wild-type AAV capsid. An rAAV virion of the present disclosure comprises: a) variant capsid protein that confers on the rAAV virion an increased ability to infect macrophages, compared to a control AAV virion comprising a wild-type AAV capsid; and b) a heterologous nucleic acid comprising a nucleotide sequence(s) encoding one or more heterologous gene products.

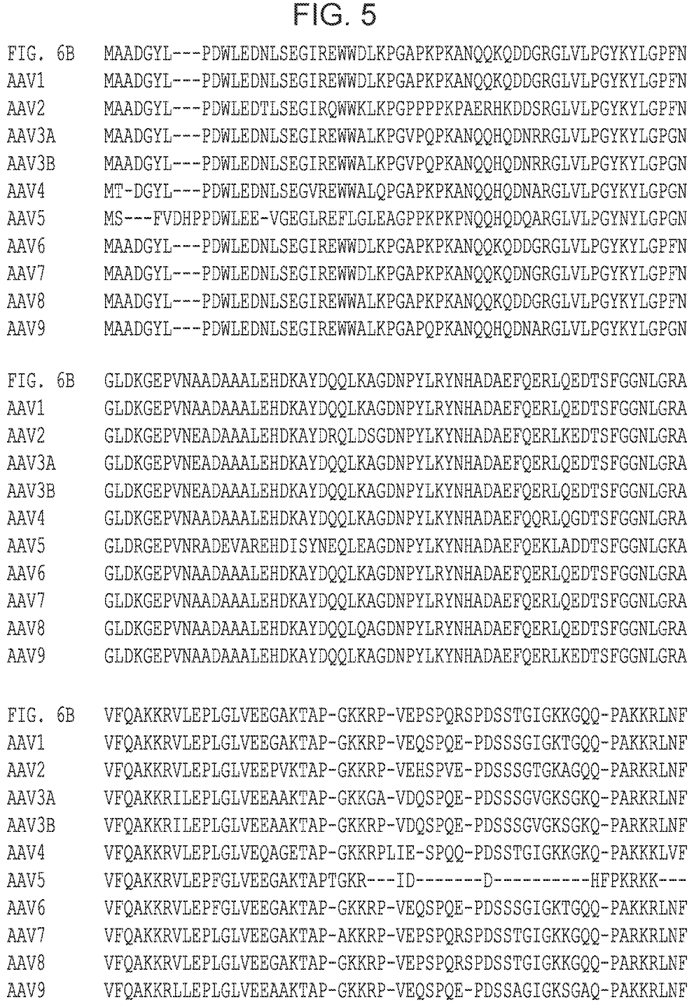

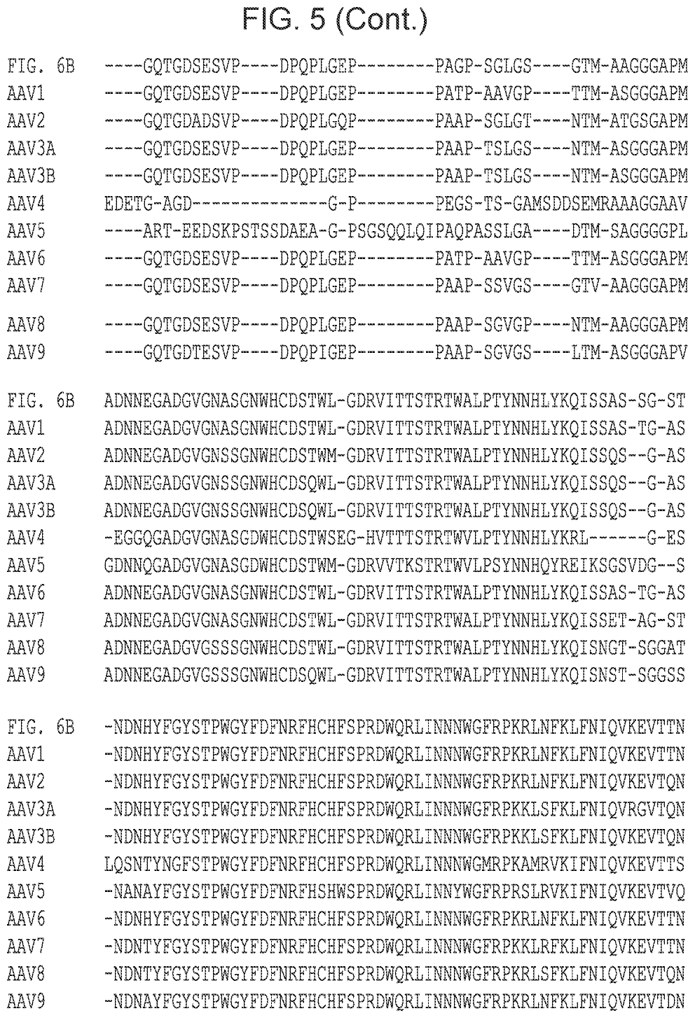

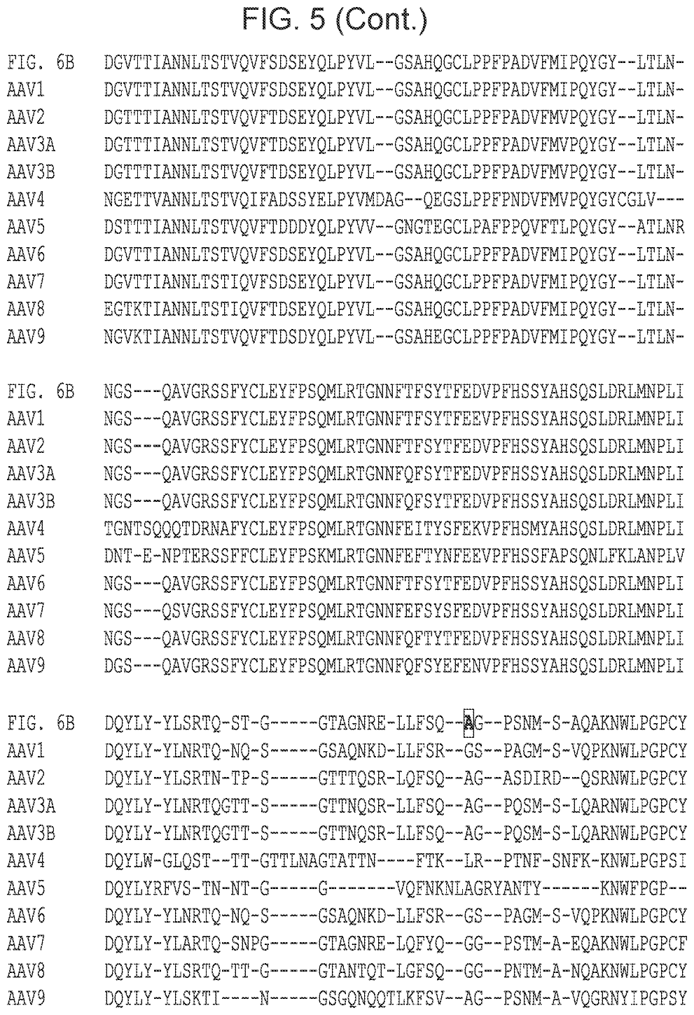

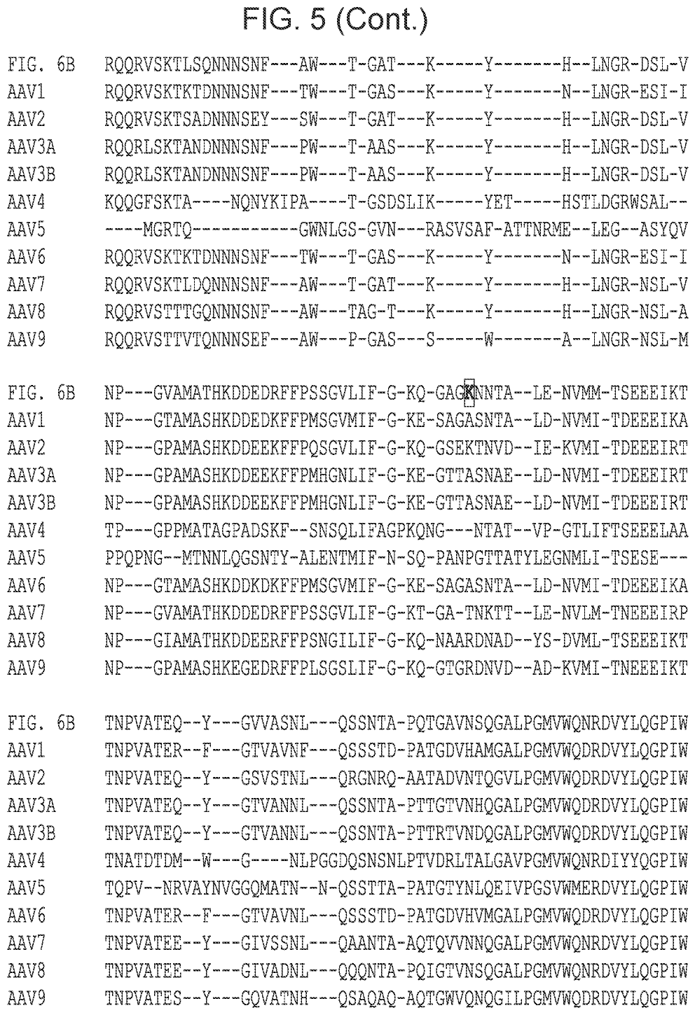

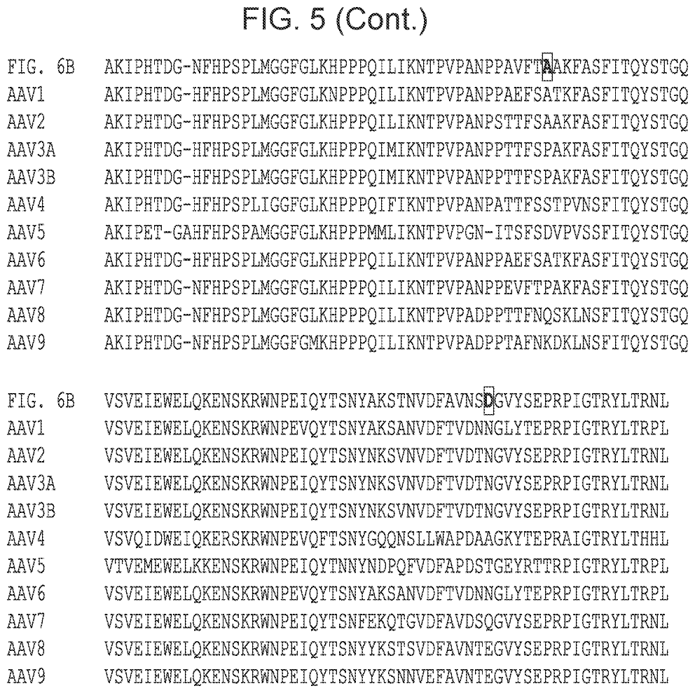

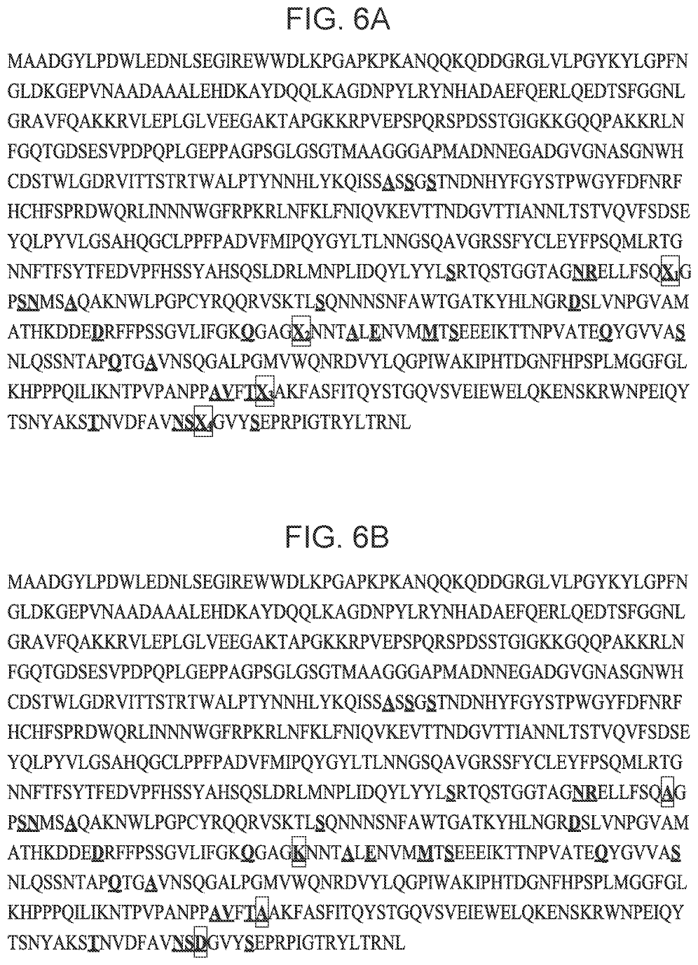

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6A, where the variant capsid protein comprises amino acid substitutions at positions 467, 551, 665, and 719 (represented in FIG. 6A by X₁, X₂, X₃, and X₄, respectively), relative to the amino acid sequence depicted in FIG. 6A, or a corresponding position in another AAV serotype. FIG. 5 provides an amino acid sequence alignment of AAV capsid proteins of various AAV serotypes, compared to the variant capsid polypeptide of FIG. 6B. Those skilled in the art can readily identify amino acid positions corresponding to positions 467, 551, 665, and 719 in other AAV serotypes such as AAV1, AAV2, AAV3, AAV4, AAV5, AAV6, AAV7, AAV8, and AAV9.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6A, where X₁ is an amino acid other than Gly; X₂ is an amino acid other than Ala; X₃ is an amino acid other than Pro; and X₄ is an amino acid other than Glu.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6A, where X₁ is selected from Ala, Arg, Asn, Asp, Cys, Gln, Glu, His, Ile, Leu, Lys, Met, Phe, Pro, Ser, Thr, Trp, Tyr, and Val; X₂ is selected from Arg, Asn, Asp, Cys, Gln, Glu, Gly, His, Ile, Leu, Lys, Met, Phe, Pro, Ser, Thr, Trp, Tyr, and Val; X₃ is selected from Ala, Arg, Asn, Asp, Cys, Gln, Glu, Gly, His, Ile, Leu, Lys, Met, Phe, Ser, Thr, Trp, Tyr, and Val; and X₄ is selected from Ala, Arg, Asn, Asp, Cys, Gln, Gly, His, Ile, Leu, Lys, Met, Phe, Pro, Ser, Thr, Trp, Tyr, and Val.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is selected from Ala, Val, Ile, and Leu; the amino acid at position 551 is selected from Lys, Arg, and His; the amino acid at position 665 is selected from Ala, Val, Ile, and Leu; and the amino acid at position 719 is Asp.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is an Ala, the amino acid at position 551 is a Lys, the amino acid at position 665 is an Ala, and the amino acid at position 719 is an Asp.

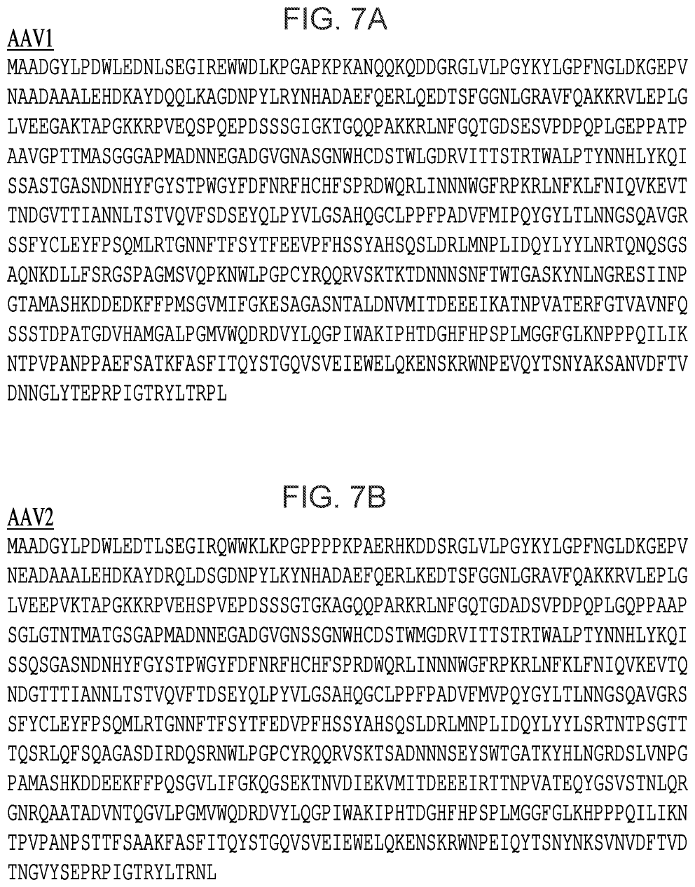

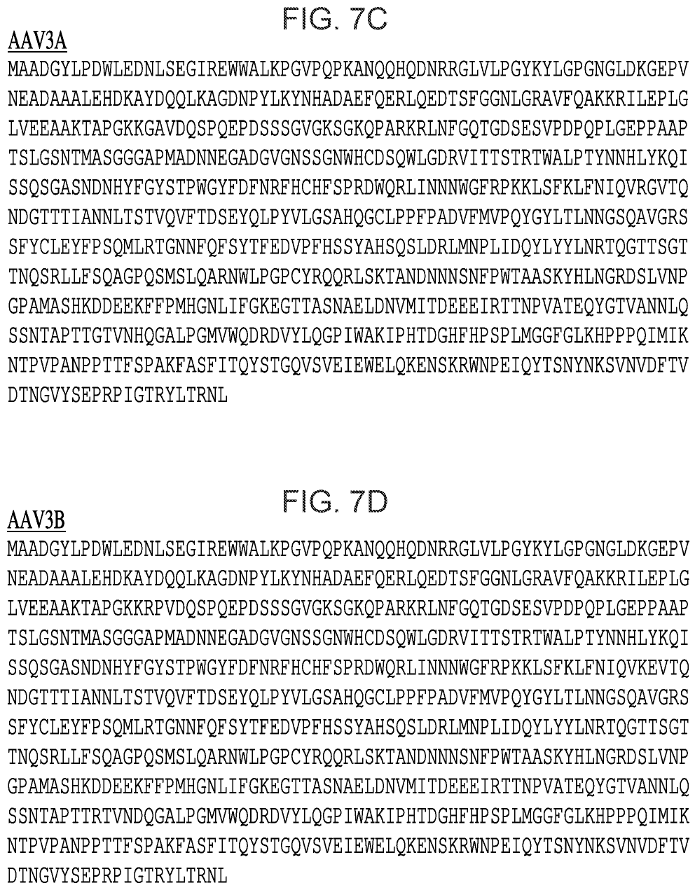

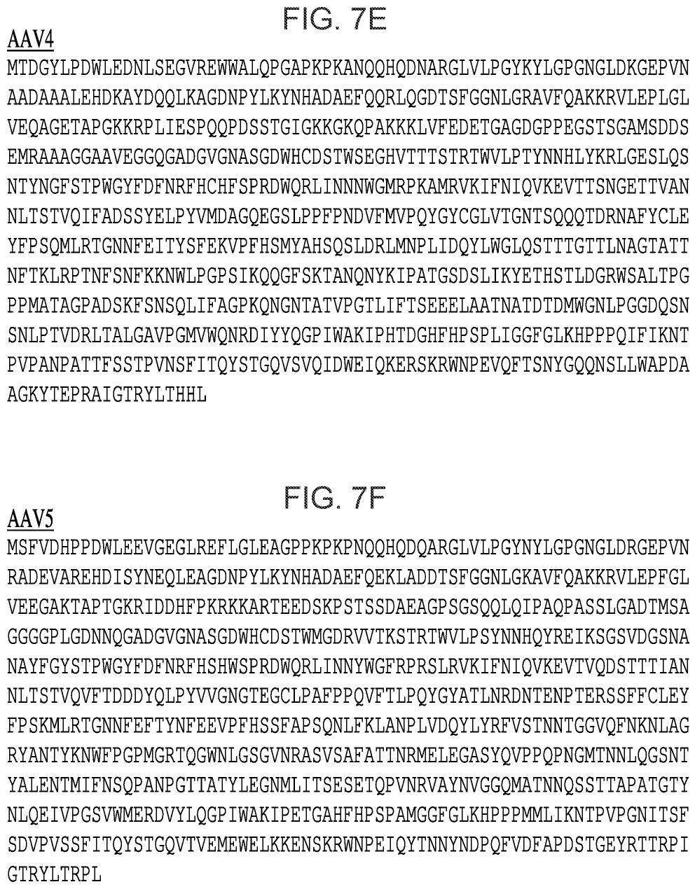

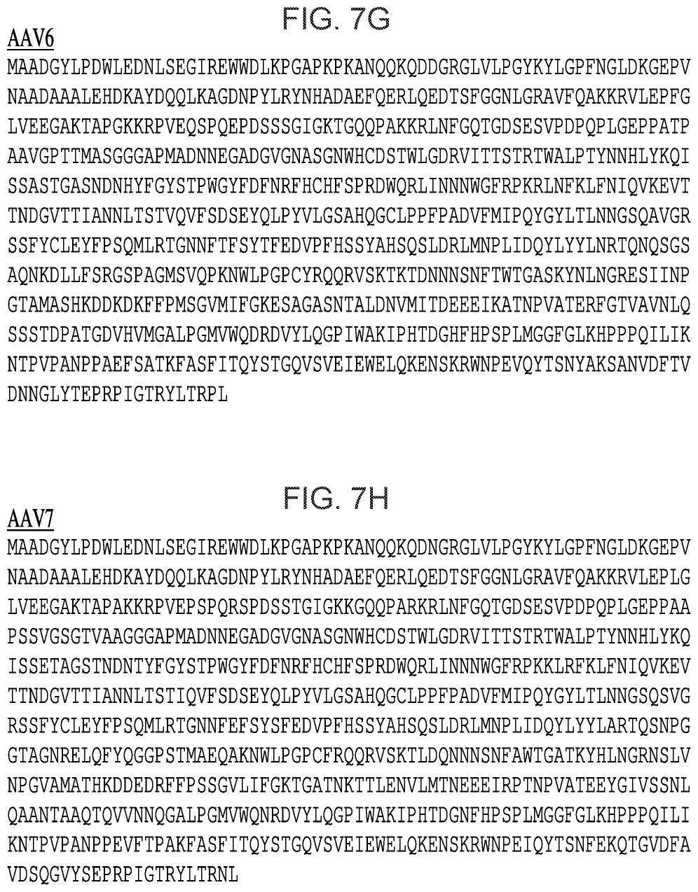

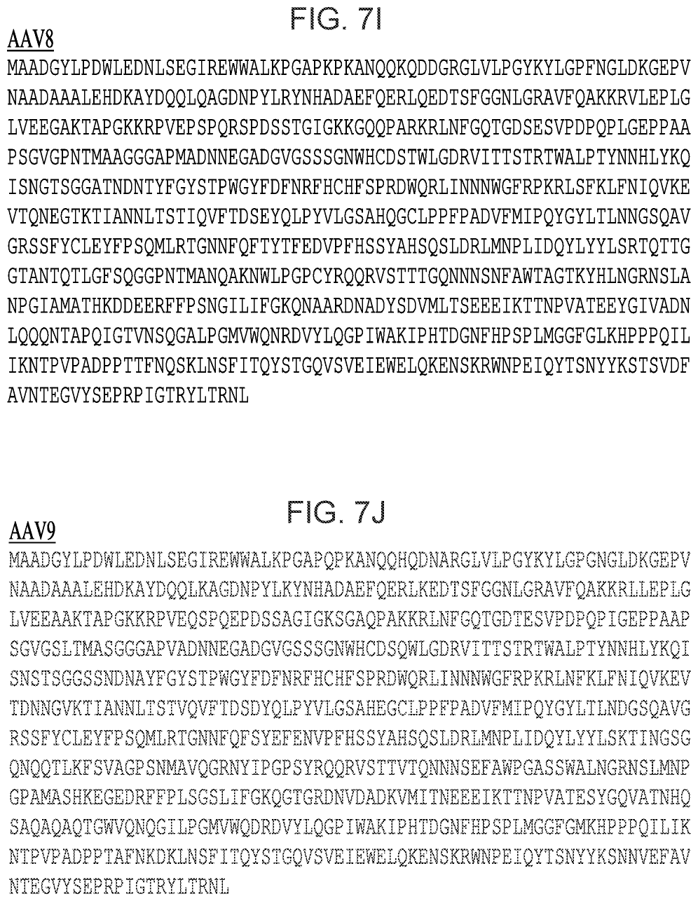

In some cases, a variant capsid protein present in an rAAV virion of the present disclosure comprises an amino acid sequence having at least about 80%, at least about 85%, at least about 90%, at least about 95%, at least about 98%, or at least about 99%, to any one of the amino acid sequences depicted in FIG. 7A-7J, where the amino acid at position 467 is an amino acid other than Gly, the amino acid at position 551 is an amino acid other than Ala, the amino acid at position 665 is an amino acid other than Pro, and the amino acid at position 719 is an amino acid other than Glu, compared to the amino acid numbering of the amino acid sequence depicted in FIG. 6A.

In some cases, a variant capsid protein present in an rAAV virion of the present disclosure comprises an amino acid sequence having at least about 80%, at least about 85%, at least about 90%, at least about 95%, at least about 98%, or at least about 99%, to any one of the amino acid sequences depicted in FIG. 7A-7J, where the amino acid at position 467 is an Ala, the amino acid at position 551 is a Lys, the amino acid at position 665 is an Ala, and the amino acid at position 719 is an Asp, compared to the amino acid numbering of the amino acid sequence depicted in FIG. 6A.

In some cases, a variant capsid protein present in an rAAV virion of the present disclosure comprises an amino acid sequence having at least about 80%, at least about 85%, at least about 90%, at least about 95%, at least about 98%, or at least about 99%, to any one of the amino acid sequences depicted in FIG. 7A-7J, where the amino acid at position 467 is an Ala, the amino acid at position 551 is a Lys, the amino acid at position 665 is an Ala, and the amino acid at position 719 is an Asp; and where amino acid at position 264 is a T, Q, or A, position 448 is an S or A, position 459 is a T or N, position 470 is an S or A, position 495 is an S or T, position 533 is a D or E, position 547 is a Q, E, or T, position 555 is a T or A, position 557 is an E or D, position 561 is an M, L, or I, position 563 is an S or N, position 593 is an A, Q, or V, position 596 is an A or T, position 661 is an A, E, or T, position 662 is a V, T, or A, position 664 is a T or S, position 718 is an N or S, and position 723 is an S or T; and where the amino acid numbering is based on the amino acid number of the amino acid sequence depicted in FIG. 6A or FIG. 6B.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is an Ala, the amino acid at position 551 is a Lys, the amino acid at position 665 is an Ala, and the amino acid at position 719 is an Asp; and where amino acid at position 264 is a T, Q, or A, position 448 is an S or A, position 459 is a T or N, position 470 is an S or A, position 495 is an S or T, position 533 is a D or E, position 547 is a Q, E, or T, position 555 is a T or A, position 557 is an E or D, position 561 is an M, L, or I, position 563 is an S or N, position 593 is an A, Q, or V, position 596 is an A or T, position 661 is an A, E, or T, position 662 is a V, T, or A, position 664 is a T or S, position 718 is an N or S, and position 723 is an S or T; or a corresponding amino acid in an amino acid sequence depicted in any one of FIG. 7A-7J.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is an Ala, the amino acid at position 551 is a Lys, the amino acid at position 665 is an Ala, and the amino acid at position 719 is an Asp; and where the amino acids at positions 264, 448, 459, 470, 495, 533, 547, 555, 557, 561, 563, 593, 596, 661, 662, 664, 718 and 723 are A, A, N, S, S, D, E, A, E, L, N, A, A, A, T, T, N, and S, respectively.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is an Ala, the amino acid at position 551 is a Lys, the amino acid at position 665 is an Ala, and the amino acid at position 719 is an Asp; and where the amino acids at positions 264, 448, 459, 470, 495, 533, 547, 555, 557, 561, 563, 593, 596, 661, 662, 664, 718 and 723 are Q, S, N, A, S, E, Q, T, D, M, S, Q, T, A, V, S, S, and S, respectively.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is an Ala, the amino acid at position 551 is a Lys, the amino acid at position 665 is an Ala, and the amino acid at position 719 is an Asp; and where the amino acids at positions 264, 448, 459, 470, 495, 533, 547, 555, 557, 561, 563, 593, 596, 661, 662, 664, 718 and 723 are A, A, T, S, T, D, Q, A, D, I, N, A, T, T, V, S, S, and T, respectively.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 6B, where the amino acid at position 467 is an Ala, the amino acid at position 551 is a Lys, the amino acid at position 665 is an Ala, and the amino acid at position 719 is an Asp; and where the amino acids at positions 264, 448, 459, 470, 495, 533, 547, 555, 557, 561, 563, 593, 596, 661, 662, 664, 718 and 723 are A, S, N, S, S, D, Q, A, E, M, S, Q, A, A, V, T, S, and S, respectively.

In some cases, the variant capsid protein present in an rAAV virion of the present disclosure comprises an amino acid sequence having at least 95%, e.g., at least 96%, at least 97%, at least 97.5%, at least 98%, at least 98.5%, or at least 99%, amino acid sequence identity to the amino acid sequence depicted in FIG. 6B, wherein the amino acids at positions 264, 266, 268, 448, 459, 460, 467, 470, 471, 474, 495, 516, 533, 547, 551, 555, 557, 561, 563, 577, 583, 593, 596, 661, 662, 664, 665, 710, 717, 718, 719, and 723 are as set out in Table 1, below.

TABLE 1

| Position | Residue |
|---|---|
| 264 | A |
| 266 | S |
| 268 | S |
| 448 | S |
| 459 | N |
| 460 | R |
| 467 | A |
| 470 | S |
| 471 | N |
| 474 | A |
| 495 | S |
| 516 | D |
| 533 | D |
| 547 | Q |
| 551 | K |
| 555 | A |
| 557 | E |
| 561 | M |
| 563 | S |
| 577 | Q |
| 583 | S |
| 593 | Q |
| 596 | A |
| 661 | A |
| 662 | V |
| 664 | T |
| 665 | A |
| 710 | T |
| 717 | N |
| 718 | S |
| 719 | D |
| 723 | S |

In some cases, the variant capsid protein present in an rAAV virion of the present disclosure comprises the amino acid sequence depicted in FIG. 6B.

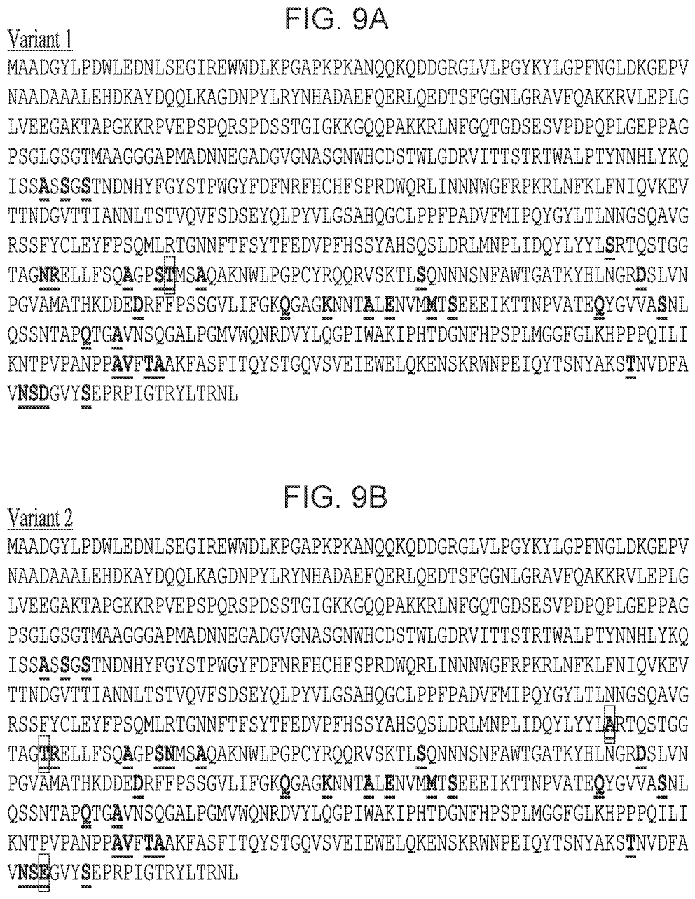

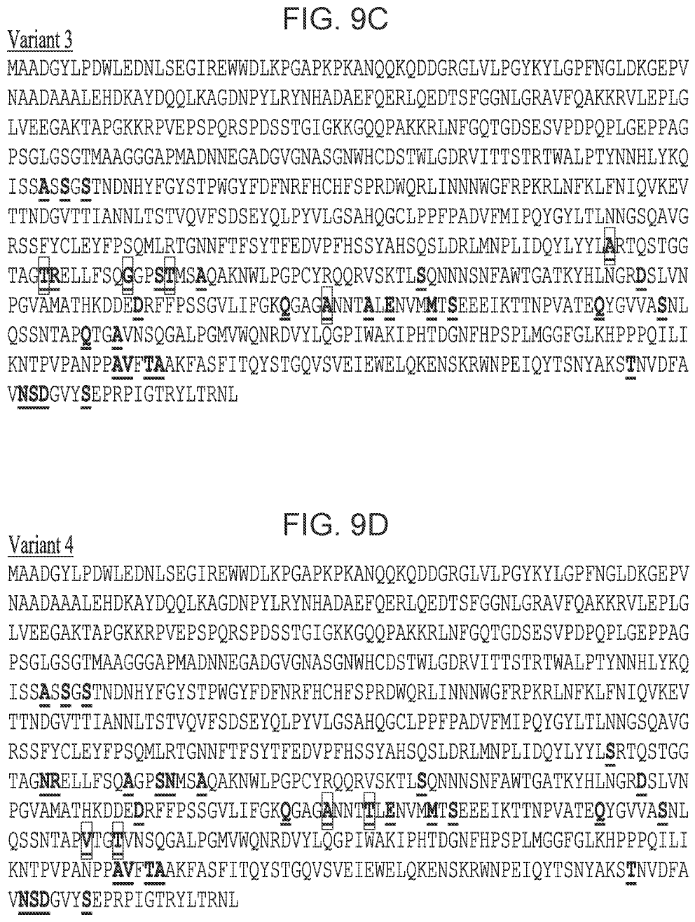

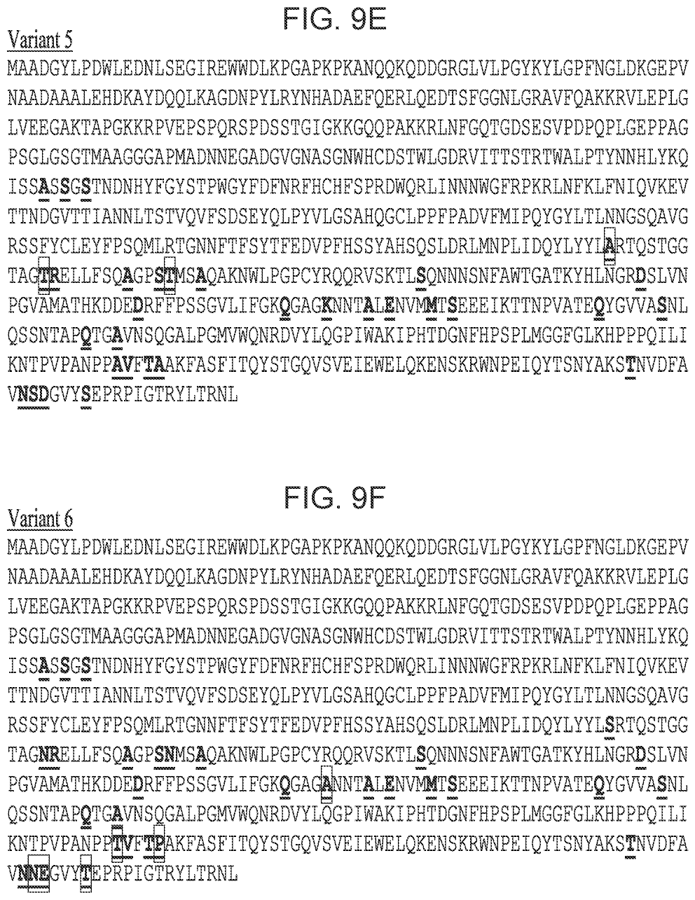

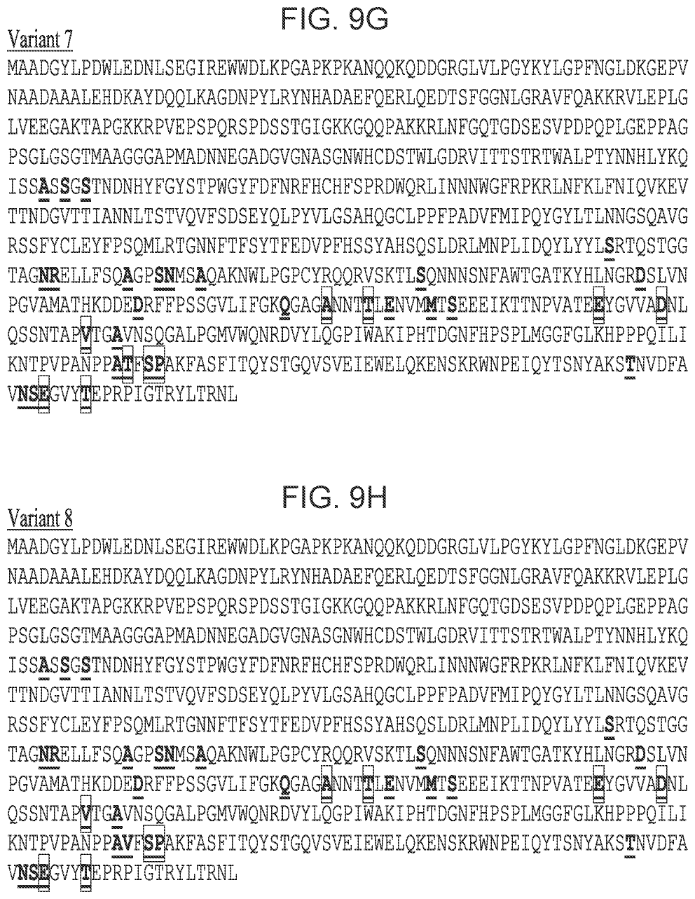

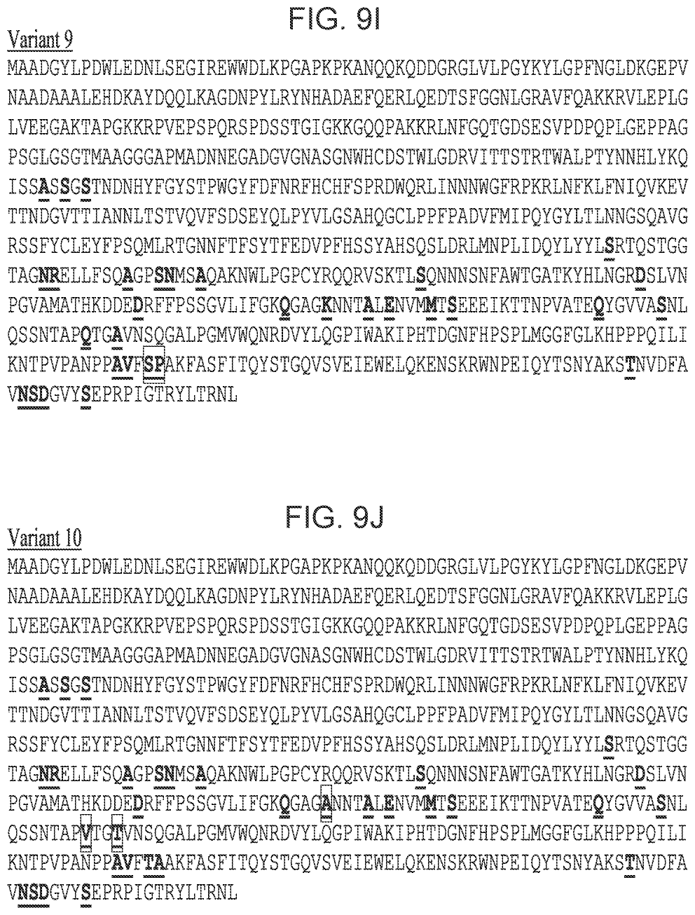

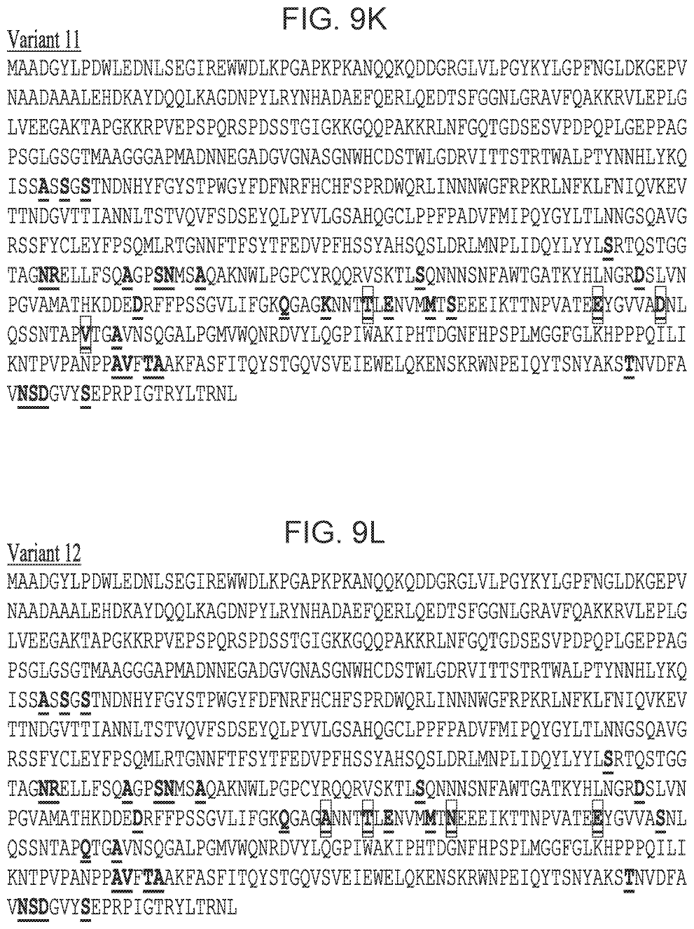

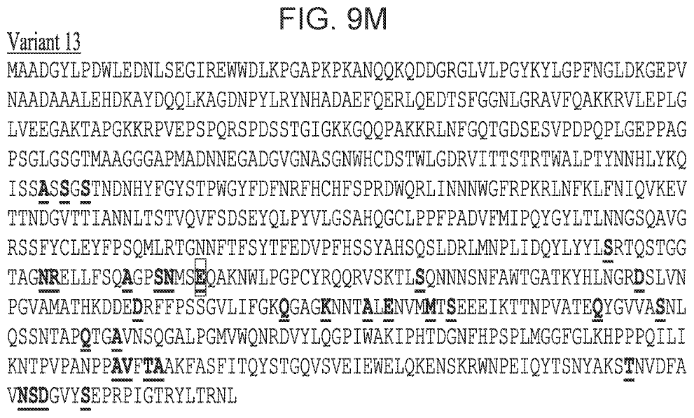

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9A, where amino acid 471 is other than Asn. In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9A, where amino acid 471 is Thr. In some cases, a variant capsid protein present in an rAAV virion of the present disclosure comprises the amino acid sequence depicted in FIG. 9A.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9B, where amino acid 448 is Ala, amino acid 459 is Thr, and amino acid 719 is Glu. In some cases, a variant capsid protein present in an rAAV virion of the present disclosure comprises the amino acid sequence depicted in FIG. 9B.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9C, where amino acid 448 is Ala, amino acid 459 is Thr, amino acid 467 is Gly, amino acid 471 is other than Asn (e.g., amino acid 471 is Ala, Arg, Asp, Cys, Gln, Glu, Gly, His, Ile, Leu, Lys, Met, Phe, Pro, Ser, Thr, Trp, Tyr, or Val), and amino acid 551 is Ala. In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9C, where amino acid 448 is Ala, amino acid 459 is Thr, amino acid 467 is Gly, amino acid 471 is Thr, and amino acid 551 is Ala. In some cases, a variant capsid protein present in an rAAV virion of the present disclosure comprises the amino acid sequence depicted in FIG. 9C.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9D, where amino acid 551 is Ala, amino acid 555 is Thr, amino acid 593 is other than Gln or Ala (e.g., amino acid 593 is Arg, Asn, Asp, Cys, Glu, Gly, His, Ile, Leu, Lys, Met, Phe, Pro, Ser, Thr, Trp, Tyr, and Val), and amino acid 596 is Thr. In some cases, a variant capsid protein present in an rAAV virion of the present disclosure comprises an amino acid sequence having at least about 80%, at least about 85%. at least about 90%, at least about 95%, at least about 98%, or at least about 99%, to the amino acid sequence depicted in FIG. 9D, where amino acid 551 is Ala, amino acid 555 is Thr, amino acid 593 is Val, and amino acid 596 is Thr. In some cases, a variant capsid protein present in an rAAV virion of the present disclosure comprises the amino acid sequence depicted in FIG. 9D.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9E, where amino acid 448 is Ala, amino acid 459 is Thr, and amino acid 471 is Thr. In some cases, a variant capsid protein present in an rAAV virion of the present disclosure comprises the amino acid sequence depicted in FIG. 9E.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9F, where amino acid 551 is Ala, amino acid 661 is Thr, amino acid 665 is Pro, amino acid 718 is other than Ser (e.g., where amino acid 718 is Ala, Arg, Asn, Asp, Cys, Gln, Glu, Gly, His, Ile, Leu, Lys, Met, Phe, Pro, Thr, Trp, Tyr, or Val), amino acid 719 is Glu, and amino acid 723 is Thr. In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9F, where amino acid 551 is Ala, amino acid 661 is Thr, amino acid 665 is Pro, amino acid 718 is Asn, amino acid 719 is Glu, and amino acid 723 is Thr. In some cases, a variant capsid protein present in an rAAV virion of the present disclosure comprises the amino acid sequence depicted in FIG. 9F.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9G, where amino acid 551 is Ala, amino acid 555 is Thr, amino acid 577 is Glu, amino acid 583 is other than Ser (e.g., amino acid 583 is Ala, Arg, Asn, Asp, Cys, Gln, Glu, Gly, His, Ile, Leu, Lys, Met, Phe, Pro, Thr, Trp, Tyr, or Val), amino acid 593 is other than Gln or Ala (e.g., amino acid 593 is Arg, Asn, Asp, Cys, Glu, Gly, His, Ile, Leu, Lys, Met, Phe, Pro, Ser, Thr, Trp, Tyr, Val), amino acid 662 is Thr, amino acid 664 is Ser, amino acid 665 is Pro, amino acid 719 is Glu, and amino acid 723 is Thr. In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9G, where amino acid 551 is Ala, amino acid 555 is Thr, amino acid 577 is Glu, amino acid 583 is Asp, amino acid 593 is Val, amino acid 662 is Thr, amino acid 664 is Ser, amino acid 665 is Pro, amino acid 719 is Glu, and amino acid 723 is Thr. In some cases, a variant capsid protein present in an rAAV virion of the present disclosure comprises the amino acid sequence depicted in FIG. 9G.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9G, where amino acid 551 is Ala, amino acid 555 is Thr, amino acid 577 is other than Gln (e.g., amino acid 577 is Ala, Arg, Asn, Asp, Cys, Glu, Gly, His, Ile, Leu, Lys, Met, Phe, Pro, Ser, Thr, Trp, Tyr, or Val), amino acid 583 is other than Ser (e.g., amino acid 583 is Ala, Arg, Asn, Asp, Cys, Gln, Glu, Gly, His, Ile, Leu, Lys, Met, Phe, Pro, Thr, Trp, Tyr, or Val), amino acid 593 is other than Gln or Ala (e.g., amino acid 593 is Arg, Asn, Asp, Cys, Glu, Gly, His, Ile, Leu, Lys, Met, Phe, Pro, Ser, Thr, Trp, Tyr, or Val), amino acid 664 is Ser, amino acid 665 is Pro, amino acid 719 is Glu, and amino acid 723 is Thr. In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9G, where amino acid 551 is Ala, amino acid 555 is Thr, amino acid 577 is Glu, amino acid 583 is Asp, amino acid 593 is Val, amino acid 664 is Ser, amino acid 665 is Pro, amino acid 719 is Glu, and amino acid 723 is Thr. In some cases, a variant capsid protein present in an rAAV virion of the present disclosure comprises the amino acid sequence depicted in FIG. 9H.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9I, where amino acid 664 is Ser, and amino acid 665 is Pro. In some cases, a variant capsid protein present in an rAAV virion of the present disclosure comprises the amino acid sequence depicted in FIG. 9I.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9J, where amino acid 551 is Ala, amino acid 593 is other than Gln or Ala (e.g., amino acid 593 is Arg, Asn, Asp, Cys, Glu, Gly, His, Ile, Leu, Lys, Met, Phe, Pro, Ser, Thr, Trp, Tyr, or Val), and amino acid 596 is Thr. In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9J, where amino acid 551 is Ala, amino acid 593 is Val, and amino acid 596 is Thr. In some cases, a variant capsid protein present in an rAAV virion of the present disclosure comprises the amino acid sequence depicted in FIG. 9J.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9K, where amino acid 555 is Thr, amino acid 577 is other than Gln (e.g., amino acid 577 is Ala, Arg, Asn, Asp, Cys, Glu, Gly, His, Ile, Leu, Lys, Met, Phe, Pro, Ser, Thr, Trp, Tyr, or Val), amino acid 583 is other than Ser (e.g., where amino acid 583 is Ala, Arg, Asn, Asp, Cys, Gln, Glu, Gly, His, Ile, Leu, Lys, Met, Phe, Pro, Thr, Trp, Tyr, or Val), and amino acid 593 is other than Gln or Ala (e.g., amino acid 593 is Arg, Asn, Asp, Cys, Glu, Gly, His, Ile, Leu, Lys, Met, Phe, Pro, Ser, Thr, Trp, Tyr, or Val). In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9K, where amino acid 555 is Thr, amino acid 577 is Glu, amino acid 583 is Asp, and amino acid 593 is Val. In some cases, a variant capsid protein present in an rAAV virion of the present disclosure comprises the amino acid sequence depicted in FIG. 9K.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9L, where amino acid 551 is Ala, amino acid 555 is Thr, amino acid 563 is Asn, and amino acid 577 is other than Gln (e.g., amino acid 577 is Ala, Arg, Asn, Asp, Cys, Glu, Gly, His, Ile, Leu, Lys, Met, Phe, Pro, Ser, Thr, Trp, Tyr, or Val). In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9L, where amino acid 551 is Ala, amino acid 555 is Thr, amino acid 563 is Asn, and amino acid 577 is Glu. In some cases, a variant capsid protein present in an rAAV virion of the present disclosure comprises the amino acid sequence depicted in FIG. 9L.

In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9M, where amino acid 474 is other than Ala (e.g., where amino acid 474 is Arg, Asn, Asp, Cys, Gln, Glu, Gly, His, Ile, Leu, Lys, Met, Phe, Pro, Ser, Thr, Trp, Tyr, or Val). In some cases, a variant capsid protein present in an rAAV virion of the present disclosure comprises an amino acid sequence having at least about 80%, at least about 85%, at least about 90%, at least about 95%, at least about 98%, or at least about 99%, to the amino acid sequence depicted in FIG. 9M, where amino acid 474 is Glu. In some cases, a variant capsid protein present in an rAAV virion of the present disclosure comprises the amino acid sequence depicted in FIG. 9M.

### Functional Characteristics

As noted above, an rAAV virion of the present disclosure comprises a variant capsid protein that confers on the rAAV virion an increased ability to infect macrophages, compared to a control AAV virion comprising a wild-type AAV capsid.

Macrophages include, e.g., adipose tissue macrophages, monocytes, Kupffer cells, sinus histiocytes, alveolar macrophages, tissue macrophages (histiocytes), Hofbauer cells, intraglomerular mesangial cells, osteoclasts, epitheloid cells, red pulp macrophages, peritoneal macrophages, LysoMac, and microglia. Macrophages can be identified, for example, using flow cytometry or immunohistochemical staining by their specific expression of one or more of the following: CD45, CD80, CD40, CD11b, CD64, F4/80 (mice)/EMR1 (human), lysozyme M, TREM-2b, CX3CR1, CCR9, MAC-1/MAC-3, and CD68.

In some cases, a variant AAV capsid protein, when present in an rAAV virion of the present disclosure, confers increased infectivity of a macrophage, compared to the infectivity of the macrophage by a control rAAV virion comprising a wild-type AAV capsid, e.g., compared to the infectivity of the macrophage by a control rAAV virion comprising a wild-type AAV capsid of AAV1, AAV2, AAV3, AAV4, AAV5, AAV6, AAV7, AAV8, or AAV9. For example, in some cases, a variant AAV capsid protein, when present in an rAAV virion of the present disclosure, confers at least 1.5-fold, at least 2-fold, at least 2.5-fold, at least 5-fold, at least 10-fold, at least 15-fold, at least 20-fold, at least 25-fold, at least 50-fold, or more than 50-fold, greater infectivity of a macrophage, compared to the infectivity of the macrophage by a control rAAV virion comprising a wild-type AAV capsid, e.g., compared to the infectivity of the macrophage by a control rAAV virion comprising a wild-type AAV capsid of AAV1, AAV2, AAV3, AAV4, AAV5, AAV6, AAV7, AAV8, or AAV9.

In some cases, a variant AAV capsid protein, when present in an rAAV virion of the present disclosure, confers increased infectivity of a microglial cell, compared to the infectivity of the microglial cell by a control rAAV virion comprising a wild-type AAV capsid, e.g., compared to the infectivity of the microglial cell by a control rAAV virion comprising a wild-type AAV capsid of AAV1, AAV2, AAV3, AAV4, AAV5, AAV6, AAV7, AAV8, or AAV9. For example, in some cases, a variant AAV capsid protein, when present in an rAAV virion of the present disclosure, confers at least 1.5-fold, at least 2-fold, at least 2.5-fold, at least 5-fold, at least 10-fold, at least 15-fold, at least 20-fold, at least 25-fold, at least 50-fold, or more than 50-fold, greater infectivity of a microglial cell, compared to the infectivity of the microglial cell by a control rAAV virion comprising a wild-type AAV capsid, e.g., compared to the infectivity of the microglial cell by a control rAAV virion comprising a wild-type AAV capsid of AAV1, AAV2, AAV3, AAV4, AAV5, AAV6, AAV7, AAV8, or AAV9.

### Heterologous Gene Products

As noted above, an rAAV virion of the present disclosure comprises: a) variant capsid protein that confers on the rAAV virion an increased ability to infect macrophages, compared to a control AAV virion comprising a wild-type AAV capsid; and b) a heterologous nucleic acid comprising a nucleotide sequence(s) encoding one or more heterologous gene products.

An rAAV virion of the present disclosure comprises a heterologous nucleic acid comprising a nucleotide sequence encoding one or more gene products (one or more heterologous gene products). In some cases, the gene product is a polypeptide. In some cases, the gene product is an RNA. In some cases, an rAAV virion of the present disclosure comprises a heterologous nucleotide sequence encoding both a heterologous nucleic acid gene product and a heterologous polypeptide gene product. Where the gene product is an RNA, in some cases, the RNA gene product encodes a polypeptide. Where the gene product is an RNA, in some cases, the RNA gene product does not encode a polypeptide. In some cases, an rAAV virion of the present disclosure comprises a single heterologous nucleic acid comprising a nucleotide sequence encoding a single heterologous gene product. In some cases, an rAAV virion of the present disclosure comprises a single heterologous nucleic acid comprising a nucleotide sequence encoding two heterologous gene products. Where the single heterologous nucleic acid encodes two heterologous gene products, in some cases, nucleotide sequences encoding the two heterologous gene products are operably linked to the same promoter. Where the single heterologous nucleic acid encodes two heterologous gene products, in some cases, nucleotide sequences encoding the two heterologous gene products are operably linked to two different promoters. In some cases, an rAAV virion of the present disclosure comprises a single heterologous nucleic acid comprising a nucleotide sequence encoding three heterologous gene products. Where the single heterologous nucleic acid encodes three heterologous gene products, in some cases, nucleotide sequences encoding the three heterologous gene products are operably linked to the same promoter. Where the single heterologous nucleic acid encodes three heterologous gene products, in some cases, nucleotide sequences encoding the three heterologous gene products are operably linked to two or three different promoters. In some cases, an rAAV virion of the present disclosure comprises two heterologous nucleic acids, each comprising a nucleotide sequence encoding a heterologous gene product.

In some cases, the one or more heterologous gene products are RNAs. In some cases, the one or more heterologous gene products are polypeptides. In some cases, the one or more heterologous gene products include both an RNA and a polypeptide. In some cases, the one or more heterologous gene products include two or more polypeptides. In some cases, the one or more heterologous gene products include two or more RNAs.

Suitable heterologous gene products include chimeric antigen receptors (CARs); engineered nucleases; CRISPR/Cas effector polypeptides; inhibitory RNAs; anti-inflammatory antibodies; and the like.

In some cases, a suitable heterologous gene product is one that provides for inhibition of microglial activation in the central nervous system (CNS) of a mammalian subject. In some cases, a suitable heterologous gene product is one that provides for reduced production of inflammatory polypeptides, such as tumor necrosis factor-alpha (TNF-α) or interleukin-6 (IL-6), by a microglial cell in the CNS. In some cases, a suitable heterologous gene product is one that provides for an increased production by a microglial cell of CX3CR1 and/or TGF-β. In some cases, a suitable heterologous gene product is one that provides for reduced expression by a microglial cell of CD74 and/or H2-AB1. In some cases, the heterologous gene produce is an anti-inflammatory antibody. Anti-inflammatory antibodies are known in the art; see, e.g., Lu et al. (2020) J. Biomedical Science 27:1.

### Antibodies

Suitable heterologous gene products include antibodies specific for a pro-inflammatory polypeptide. Suitable heterologous gene products include antibodies specific for AOC3 (VAP-1), CAM-3001, CCL11 (cotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), myostatin, OX-40, scleroscin, SOST, TGF beta 1, TNF-α, or VEGF-A.

Suitable heterologous gene products include, e.g., an anti-TNF-α antibody; an anti-CD3 antibody; an anti-IL6 antibody; a soluble TNF receptor (TNFR; e.g., a soluble TNFR fused to an immunoglobulin (Ig) Fc polypeptide); an anti-IL-1 receptor antibody (e.g., an anti-IL1β antibody); an anti-CD20 antibody; an anti-IL-17 antibody (e.g., Ixekizumab); an anti-IL-12/23 antibody.

In some cases, the heterologous gene product is an anti-TNFα antibody. In some cases, the heterologous gene product is an anti-TNFα antibody. Anti-TNFα antibodies are known in the art; and any such anti-TNFα antibody can be encoded by a heterologous nucleic acid in an rAAV virion of the present disclosure. See, e.g., Hu et al. (2013) J. Biol. Chem. 288:27059; U.S. Pat. No. 8,216,583. In some cases, an anti-TNFα antibody comprises complementarity-determining regions (CDRs) present in an anti-TNFα antibody having the following amino acid sequence:
EVOLVESGGGLVQPGRSLRLSCAASGFTFDDYAMHWVRQAPGKGLEWVSAITWNSGHIDYAD SVEGRFTISRDNAKNSLYLQMNSLRAEDTAVYYCAKVSYLSTASSLDYWGQGTLVTVSSDIQM TQSPSSLSASVGDRVTITCRASQGIRNYLAWYQQKPGKAPKLLIYAASTLQSGVPSRFSGSGSGT DFTLTISSLQPEDVATYYCQRYNRAPYTFGQGTKVEIKR (SEQ ID NO:1). For example, an anti-TNFα antibody can comprise: a) heavy chain variable region (VH) CDRs having the following amino acid sequences: i) CDR1: DYAMH (SEQ ID NO:2); ii) CDR2: AITWNSGHIDYADSVEG (SEQ ID NO:3); and iii) CDR3: VSYLSTASSLDY (SEQ ID NO:4); and b) light chain variable region (VL) CDRs having the following amino acid sequences: i) CDR1: RASQGIRNYLA (SEQ ID NO:5); ii) CDR2: AASTLQS (SEQ ID NO:6); and iii) CDR3: QRYNRAPYT (SEQ ID NO:7).

Other suitable anti-TNFα antibodies include, e.g., certolizumab, golimumab, infliximab, and remsima. Other suitable anti-TNFα antibodies include, e.g., an anti-TNFα antibody as described in U.S. Pat. No. 10,787,508; and the like.

In some cases, the heterologous gene product is an anti-IL-6 antibody. Anti-IL-6 antibodies are known in the art; and any such anti-IL-6 antibody can be encoded by a heterologous nucleic acid in an rAAV virion of the present disclosure. See, e.g., U.S. Pat. No. 10,787,511; Alten (2011) Ther. Adv. Musculoskelet. Dis. 3:133; and U.S. Pat. No. 5,795,965. Examples include Tocilizumab, Siltuximab; and the like.

In some cases, the heterologous gene product is an anti-CD3 antibody. wherein the anti-CD3 antibody comprises a heavy chain complementarity determining region 1 (CDRH1) comprising the amino acid sequence GYGMH (SEQ ID NO:8), a heavy chain complementarity determining region 2 (CDRH2) comprising the amino acid sequence VIWYDGSKKYYVDSVKG (SEQ ID NO:9), a heavy chain complementarity determining region 3 (CDRH3) comprising the amino acid sequence QMGYWHFDL (SEQ ID NO: 10), a light chain complementarity determining region 1 (CDRL1) comprising the amino acid sequence RASQSVSSYLA (SEQ ID NO:11), a light chain complementarity determining region 2 (CDRL2) comprising the amino acid sequence DASNRAT (SEQ ID NO:12), and a light chain complementarity determining region 3 (CDRL3) comprising the amino acid sequence QQRSNWPPLT (SEQ ID NO:13).

Suitable heterologous gene products include immune checkpoint inhibitors, e.g., in connection with treatment of cancer. Suitable heterologous gene products include chimeric antigen receptors (CARs), e.g., in connection with treatment of cancer.

### Chimeric Antigen Receptor

In some cases, a heterologous gene product is a CAR, where the CAR is specific for a cancer-associated antigen. Cancer-associated antigens include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

A CAR generally comprises: a) an extracellular domain comprising an antigen-binding domain (e.g., an antigen-binding polypeptide, such as a scFv or a nanobody); b) a transmembrane region; and c) a cytoplasmic domain comprising an intracellular signaling domain (intracellular signaling polypeptide). In some cases, a CAR comprises: a) an extracellular domain comprising the antigen-binding domain; b) a transmembrane region; and c) a cytoplasmic domain comprising: i) a co-stimulatory polypeptide; and ii) an intracellular signaling domain. In some cases, a CAR comprises hinge region between the extracellular antigen-binding domain and the transmembrane domain. Thus, in some cases, a CAR comprises: a) an extracellular domain comprising the antigen-binding domain; b) a hinge region; c) a transmembrane region; and d) a cytoplasmic domain comprising an intracellular signaling domain. In some cases, a CAR comprises: a) an extracellular domain comprising the antigen-binding domain; b) a hinge region; c) a transmembrane region; and d) a cytoplasmic domain comprising: i) a co-stimulatory polypeptide; and ii) an intracellular signaling domain.

Exemplary CAR structures are known in the art (See e.g., WO 2009/091826; US 20130287748; WO 2015/142675; WO 2014/055657; WO 2015/090229; and U.S. Pat. No. 9,587,020.

In some cases, a CAR is a single polypeptide chain. In some cases, a CAR comprises two polypeptide chains.

CARs specific for a variety of tumor antigens are known in the art; for example CD171-specific CARs (Park et al., Mol Ther (2007) 15(4):825-833), EGFRvIII-specific CARs (Morgan et al., Hum Gene Ther (2012) 23(10): 1043-1053), EGF-R-specific CARs (Kobold et al., J. Natl Cancer Inst (2014) 107(1):364), carbonic anhydrase IX-specific CARs (Lamers et al., Biochem Soc Trans (2016) 44(3):951-959), folate receptor-α (FR-α)-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL-13Rα2-specific CARs (Brown et al., Clin Cancer Res (2015) 21(18):4062-4072), ganglioside GD2-specific CARs (Louis et al., Blood (2011) 118(23):6050-6056; Caruana et al., Nat Med (2015) 21(5):524-529; Yu et al. (2018) J. Hematol. Oncol. 11:1), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2): 154-166), mesothelin (MSLN)-specific CARs (Moon et al, Clin Cancer Res (2011) 17(14):4719-30), NKG2D-specific CARs (VanSeggelen et al., Mol Ther (2015) 23(10): 1600-1610), CD19-specific CARs (Axicabtagene ciloleucel (Yescarta™) and Tisagenlecleucel (Kymriah™). See also, Li et al., J Hematol and Oncol (2018) 11:22, reviewing clinical trials of tumor-specific CARs; Heyman and Yan (2019) Cancers 11:pii:E191; Baybutt et al. (2019) Clin. Pharmacol. Ther. 105:71.

### Antigen-Binding Domain

As noted above, a CAR comprises an extracellular domain comprising an antigen-binding domain. The antigen-binding domain present in a CAR can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable. In some cases, the antigen-binding domain is a nanobody.

In some cases, the antigen bound by the antigen-binding domain of a CAR is selected from: a MUC1 polypeptide, an LMP2 polypeptide, an epidermal growth factor receptor (EGFR) vIII polypeptide, a HER-2/neu polypeptide, a melanoma antigen family A. 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a ber-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFB) polypeptide, a MAD-CT-2 polypeptide, or a Fos-related antigen-1 (FOSL) polypeptide. In some cases, the antigen is a human papilloma virus (HPV) antigen. In some cases, the antigen is an alpha-feto protein (AFP) antigen. In some cases, the antigen is a Wilms tumor-1 (WT1) antigen.

The antigen-binding polypeptide of a CAR can bind any of a variety of cancer-associated antigens, including, e.g., antigens of the immunoglobulin superfamily (see, e.g., Barclay (2003) Seminars in Immunology 15:215); antigens of the tumor necrosis factor (TNF) superfamily (see, e.g., Aggarwal et al. (2012) Blood 119:651; Locksley et al. (2001) Cell 104:487; and Hehlgan and Pfeffer (2005) Immunol. 115:1); antigens of the TNF receptor (TNFR) superfamily (see, e.g., Locksley et al. (2001) Cell 104:487; and Hehlgan and Pfeffer (2005) Immunol. 115:1); antigens of the B7 superfamily (see, e.g., Greenwald et al. (2005) Ann. Rev. Immunol. 23:515; and Sharpe and Freeman (2002) Nat. Rev. Immunol. 2:116); and antigens of the lectin superfamily (see, e.g., Zelensky and Gready (2005) FEBS J. 272:6179).

The antigen-binding polypeptide of a CAR can bind any of a variety of cancer-associated antigens, including, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), B-cell maturation antigen (BCMA), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

VH and VL amino acid sequences of various cancer-associated antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055.

### Hinge Region

As noted above, a CAR can include a hinge region between the extracellular domain and the transmembrane domain. As used herein, the term “hinge region” refers to a flexible polypeptide connector region (also referred to herein as “hinge” or “spacer”) providing structural flexibility and spacing to flanking polypeptide regions and can consist of natural or synthetic polypeptides. The hinge region can include complete hinge region derived from an antibody of a different class or subclass from that of the CHI domain. The term “hinge region” can also include regions derived from CD8 and other receptors that provide a similar function in providing flexibility and spacing to flanking regions.

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

As non-limiting examples, an immunoglobulin hinge region can include one of the following amino acid sequences: DKTHT (SEQ ID NO:14); CPPC (SEQ ID NO:15); CPEPKSCDTPPPCPR (SEQ ID NO:16); ELKTPLGDTTHT (SEQ ID NO:17); KSCDKTHTCP (SEQ ID NO:18); KCCVDCP (SEQ ID NO:19); KYGPPCP (SEQ ID NO:20); EPKSCDKTHTCPPCP (SEQ ID NO:21) (human IgG1 hinge); ERKCCVECPPCP (SEQ ID NO:22) (human IgG2 hinge); ELKTPLGDTTHTCPRCP (SEQ ID NO:23) (human IgG3 hinge); SPNMVPHAHHAQ (SEQ ID NO:24) (human IgG4 hinge); and the like. The hinge region can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:25), or a variant thereof.

### Transmembrane Domain

Any transmembrane (TM) domain that provides for insertion of a polypeptide into the cell membrane of a eukaryotic (e.g., mammalian) cell is suitable for use. The transmembrane region of a CAR can be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R .alpha., ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. The transmembrane domain can be synthetic, in which case it can comprise predominantly hydrophobic residues such as leucine and valine. In some cases, a triplet of phenylalanine, tryptophan and valine will be found at each end of a synthetic transmembrane domain.

As one non-limiting example, the TM sequence IYIWAPLAGTCGVLLLSLVITLYC (SEQ ID NO:26) can be used. Additional non-limiting examples of suitable TM sequences include: a) CD8 beta derived TM: LGLLVAGVLVLLVSLGVAIHLCC (SEQ ID NO:27); b) CD4 derived TM: ALIVLGGVAGLLLFIGLGIFFCVRC (SEQ ID NO:28); c) CD3 zeta derived TM: LCYLLDGILFIYGVILTALFLRV (SEQ ID NO:29); d) CD28 derived TM: WVLVVVGGVLACYSLLVTVAFIIFWV (SEQ ID NO:30); e) CD134 (OX40) derived TM: VAAILGLGLVLGLLGPLAILLALYLL (SEQ ID NO:31); and f) CD7 derived TM: ALPAALAVISFLLGLGLGVACVLA (SEQ ID NO:32).

### Intracellular Domain—Co-Stimulatory Polypeptide

The intracellular portion (cytoplasmic domain) of a CAR can comprise one or more co-stimulatory polypeptides. Non-limiting examples of suitable co-stimulatory polypeptides include, but are not limited to, 4-1BB (CD137), CD28, ICOS, OX-40, BTLA, CD27, CD30, GITR, and HVEM. Suitable co-stimulatory polypeptides include, e.g.: 1) a 4-1BB polypeptide having at least 90%, at least 95%, at least 98%, or 100%, amino acid sequence identity to the following amino acid sequence: KRGRKKLLYIFKQPFMRPVQTTQEEDGCSCRFPEEEEGGCEL (SEQ ID NO:33); 2) a CD28 polypeptide having at least 90%, at least 95%, at least 98%, or 100%, amino acid sequence identity to the following amino acid sequence:
FWVRSKRSRLLHSDYMNMTPRRPGPTRKHYQPYAPPRDFAAYRS (SEQ ID NO:34); 3) an ICOS polypeptide having at least 90%, at least 95%, at least 98%, or 100%, amino acid sequence identity to the following amino acid sequence: TKKKYSSSVHDPNGEYMFMRAVNTAKKSRLTDVTL (SEQ ID NO:35); 4) an OX40 polypeptide having at least 90%, at least 95%, at least 98%, or 100%, amino acid sequence identity to the following amino acid sequence:
RRDQRLPPDAHKPPGGGSFRTPIQEEQADAHSTLAKI (SEQ ID NO:36); 5) a BTLA polypeptide having at least 90%, at least 95%, at least 98%, or 100%, amino acid sequence identity to the following amino acid sequence:
CCLRRHQGKQNELSDTAGREINLVDAHLKSEQTEASTRQNSQVLLSETGIYDNDPDLCFRMQEG SEVYSNPCLEENKPGIVYASLNHSVIGPNSRLARNVKEAPTEYASICVRS (SEQ ID NO:37); 6) a CD27 polypeptide having at least 90%, at least 95%, at least 98%, or 100%, amino acid sequence identity to the following amino acid sequence:
HQRRKYRSNKGESPVEPAEPCRYSCPREEEGSTIPIQEDYRKPEPACSP (SEQ ID NO:38); 7) a CD30 polypeptide having at least 90%, at least 95%, at least 98%, or 100%, amino acid sequence identity to the following amino acid sequence:
RRACRKRIRQKLHLCYPVQTSQPKLELVDSRPRRSSTQLRSGASVTEPVAEERGLMSQPLMETC HSVGAAYLESLPLQDASPAGGPSSPRDLPEPRVSTEHTNNKIEKIYIMKADTVIVGTVKAELPEG RGLAGPAEPELEEELEADHTPHYPEQETEPPLGSCSDVMLSVEEEGKEDPLPTAASGK (SEQ ID NO:39); 8) a GITR polypeptide having at least 90%, at least 95%, at least 98%, or 100%, amino acid sequence identity to the following amino acid sequence:
HIWQLRSQCMWPRETQLLLEVPPSTEDARSCQFPEEERGERSAEEKGRLGDLWV (SEQ ID NO:40); and 9) an HVEM polypeptide having at least 90%, at least 95%, at least 98%, or 100%, amino acid sequence identity to the following amino acid sequence:
CVKRRKPRGDVVKVIVSVQRKRQEAEGEATVIEALQAPPDVTTVAVEETIPSFTGRSPNH (SEQ ID NO:41). The co-stimulatory polypeptide can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

### Intracellular Domain—Signaling Polypeptide

The intracellular portion of a CAR can comprise a signaling polypeptide. Suitable signaling polypeptides include, e.g., an immunoreceptor tyrosine-based activation motif (ITAM)-containing intracellular signaling polypeptide. An ITAM motif is YX₁X₂L/I (SEQ ID NO:42), where X₁ and X₂ are independently any amino acid. In some cases, the intracellular signaling domain of a subject CAR comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃),(YX₁X₂L/I) (SEQ ID NO:43), where n is an integer from 6 to 8, and each of the 6-8 X₃ can be any amino acid. In some cases, the intracellular signaling domain of a CAR comprises 3 ITAM motifs.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain).

### Immune Checkpoint Inhibitors

Exemplary immune checkpoint inhibitors include inhibitors that target immune checkpoint polypeptide such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint. In some cases, the anti-immune checkpoint antibody is a monoclonal antibody. In some cases, the anti-immune checkpoint antibody is humanized, or de-immunized such that the antibody does not substantially elicit an immune response in a human. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Antibodies, e.g., monoclonal antibodies (including scFv and nanobodies), that are specific for immune checkpoints and that function as immune checkpoint inhibitors, are known in the art. See, e.g., Wurz et al. (2016) Ther. Adv. Med. Oncol. 8:4; and Naidoo et al. (2015) Ann. Oncol. 26:2375.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CellDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof.

Suitable anti-LAG3 antibodies include, e.g., BMS-986016 and LAG525. Suitable anti-GITR antibodies include, e.g., TRX518, MK-4166, INCAGN01876, and MK-1248. Suitable anti-OX40 antibodies include, e.g., MEDI0562, INCAGN01949, GSK2831781, GSK-3174998, MOXR-0916, PF-04518600, and LAG525. Suitable anti-VISTA antibodies are provided in, e.g., WO 2015/097536.

### Anti-PD-1 Antibodies

In some cases, an immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18.

In some cases, the anti-PD1 antibody is pembrolizumab. The amino acid sequence of the heavy chain of pembrolizumab is:

(SEQ ID NO: 44)
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTTVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSF
FLYSRLTVDKSRWQEGNVFSCSVMHEALHNHYTQKSLSLSLGK.

The amino acid sequence of the heavy chain variable (VH) region is underlined.

The amino acid sequence of the light chain of pembrolizumab is:

(SEQ ID NO: 45)
EIVLTQSPATLSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC.

The amino acid sequence of the light chain variable (VL) region is underlined.

In some cases, the anti-PD-1 antibody comprises the VH and VL regions of pembrolizumab. In some cases, the anti-PD-1 antibody comprises heavy and light chain CDRs of pembrolizumab.

In some cases, the anti-PD-1 antibody is nivolumab (also known as MDX-1106 or BMS-936558; see, e.g., Topalian et al. (2012) N. Eng. J. Med. 366:2443-2454; and U.S. Pat. No. 8,008,449). The amino acid sequence of the heavy chain of nivolumab is:

(SEQ ID NO: 46)
QVQLVESGGGVVQPGRSLRLDCKASGITFSNSGMHWVRQAPGKGL
EWVAVIWYDGSKRYYADSVKGRFTISRDNSKNTLFLQMNSLRAED
TAVYYCATNDDYWGQGTLVTVSSASTKGPSVFPLAPCSRSTSEST
AALGCLVKDYFPEPVTVSWNSGALTSGVHTFPAVLQSSGLYSLSS
VVTVPSSSLGTKTYTCNVDHKPSNTKVDKRVESKYGPPCPPCPAP
EFLGGPSVFLFPPKPKDTLMISRTPEVTCVVVDVSQEDPEVQFNW
YVDGVEVHNAKTKPREEQFNSTYRVVSVLTVLHQDWLNGKEYKCK
VSNKGLPSSIEKTISKAKGQPREPQVYTLPPSQEEMTKNQVSLTC
LVKGFYPSDIAVEWESNGQPENNYKTTPPVLDSDGSFFLYSRLTV
DKSRWQEGNVFSCSVMHEALHNHYTQKSLSLSLGK.

The amino acid sequence of the light chain of nivolumab is:

(SEQ ID NO: 47)
EIVLTQSPATLSLSPGERATLSCRASQSVSSYLAWYQQKPGQAPR
LLIYDASNRATGIPARFSGSGSGTDFTLTISSLEPEDFAVYYCQQ
SSNWPRTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

In some cases, the anti-PD-1 antibody comprises heavy and light chain CDRs of nivolumab.

### Anti-CTLA4 Antibodies

In some cases, the anti-CTLA-4 antibody is ipilimumab or tremelimumab. For tremelimumab, see, e.g., Ribas et al. (2013) J. Clin. Oncol. 31:616-22.

In some cases, the anti-CTLA-4 antibody is ipilimumab. The amino acid sequence of the heavy chain of ipilimumab is:

(SEQ ID NO: 48)
QVQLVESGGGVVQPGRSLRLSCAASGFTFSSYTMHWVRQAPGKGL
EWVTFISYDGNNKYYADSVKGRFTISRDNSKNTLYLQMNSLRAED
TAIYYCARTGWLGPFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKRVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGS
FFLYSKLTVDKSRWQQGNVFSCSVMHEALHNHYTQKSLSLSPGK.

The amino acid sequence of the VH region is underlined.

The amino acid sequence of the light chain of ipilimumab is:

(SEQ ID NO: 49)
EIVLTQSPGTLSLSPGERATLSCRASQSVGSSYLAWYQQKPGQAP
RLLIYGAFSRATGIPDRESGSGSGTDFTLTISRLEPEDFAVYYCQ
QYGSSPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC.

The amino acid sequence of the VL region is underlined.

In some cases, the anti-CTLA4 antibody comprises the VH and VL regions of ipilimumab. In some cases, the anti-CTLA4 antibody comprises heavy and light chain CDRs of ipilimumab.

### Anti-PD-L1 Antibodies

In some cases, the immune checkpoint inhibitor is an anti-PD-L1 monoclonal antibody. In some cases, the anti-PD-L1 monoclonal antibody is BMS-935559, MEDI4736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

In some cases, the anti-PD-L1 antibody is atezolizumab. The amino acid sequence of the heavy chain of atezolizumab is:

(SEQ ID NO: 50)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYASTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGS
FFLYSKLTVDKSRWQQGNVFSCSVMHEALHNHYTQKSLSLSPGK.

The amino acid sequence of the light chain of atezolizumab is:

(SEQ ID NO: 51)
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

In some cases, the anti-PD-L1 antibody comprises heavy and light chain CDRs of atezolizumab.

In some cases, the anti-PDL1 antibody is KN035, a fully humanized anti-PD-L1 single domain antibody fused to a human IgG1 Fc polypeptide. Zhang et al. (2017) Cell Discov. 3:17004; and WO 2017/020801. The single-domain antibody portion of KN035 can comprise the amino acid sequence:

(SEQ ID NO: 52)
QVQLQESGGGLVQPGGSLRLSCAASGKMSSRRCMAWFRQAPGKER
ERVAKLLTTSGSTYLADSVKGRFTISQNNAKSTVYLQMNSLKPED
TAMYYCAADSFEDPTCTLVTSSGAFQYWGQGTQVTVS,

where the underlined amino acids are CDR1, CDR2, and CDR3.

### Gene-Editing Polypeptides

Suitable heterologous gene products include gene editing polypeptides. Suitable gene-editing systems include: i) a clustered regularly interspaced short palindromic repeats (CRISPR) associated (Cas) effector polypeptide and a guide nucleic acid; ii) a zinc finger nuclease (ZFN); iii) a transcription activator-like effector nuclease (TALEN); and iv) a meganuclease (e.g., an engineered meganuclease).

### CRISPR/Cas Effector Polypeptides

In some cases, the heterologous polypeptide encoded is a CRISPR/Cas effector polypeptide. In some cases, a suitable CRISPR-Cas effector polypeptide is a class 2 CRISPR/Cas effector polypeptide such as a type II, type V, or type VI CRISPR/Cas effector polypeptide. In some cases, a suitable CRISPR/Cas effector polypeptide is a class 2 CRISPR/Cas effector polypeptide. In some cases, a suitable CRISPR/Cas effector polypeptide is a type II CRISPR/Cas effector polypeptide (e.g., a Cas9 protein). In some cases, a suitable CRISPR/Cas effector polypeptide is a type V CRISPR/Cas effector polypeptide (e.g., a Cpf1 protein, a C2c1 protein, or a C2c3 protein), e.g., a Cas12a, a Cas12b, a Cas12c, a Cas12d, or a Cas12e polypeptide. In some cases, a suitable CRISPR/Cas effector polypeptide is a type VI CRISPR/Cas effector polypeptide (e.g., a C2c2 protein; also referred to as a “Cas13a” protein), e.g., a Cas13a, a Cas13b, a Cas13c, or a Cas13d polypeptide. In some cases, a suitable CRISPR/Cas effector polypeptide is a CasX protein. In some cases, a suitable CRISPR/Cas effector polypeptide is a CasY protein. In some cases, a suitable CRISPR/Cas effector polypeptide is a CasZ protein. In some cases, a suitable CRISPR/Cas effector polypeptide is a Cas14a, a Cas14b, or a Cas14c polypeptide.

In class 2 CRISPR systems, the functions of the effector complex (e.g., the cleavage of target DNA) are carried out by a single endonuclease (e.g., see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97); and Shmakov et al. (2017) Nature Reviews Microbiology 15:169. As such, the term “class 2 CRISPR/Cas protein” is used herein to encompass the CRISPR/Cas effector polypeptide (e.g., the target nucleic acid cleaving protein) from class 2 CRISPR systems. Thus, the term “class 2 CRISPR/Cas effector polypeptide” as used herein encompasses type II CRISPR/Cas effector polypeptides (e.g., Cas9); type V-A CRISPR/Cas effector polypeptides (e.g., Cpf1 (also referred to a “Cas12a”)); type V-B CRISPR/Cas effector polypeptides (e.g., C2cl (also referred to as “Cas12b”)); type V-C CRISPR/Cas effector polypeptides (e.g., C2c3 (also referred to as “Cas12c”)); type V-U1 CRISPR/Cas effector polypeptides (e.g., C2c4); type V-U2 CRISPR/Cas effector polypeptides (e.g., C2c8); type V-U5 CRISPR/Cas effector polypeptides (e.g., C2c5); type V-U4 CRISPR/Cas proteins (e.g., C2c9); type V-U3 CRISPR/Cas effector polypeptides (e.g., C2c10); type VI-A CRISPR/Cas effector polypeptides (e.g., C2c2 (also known as “Cas13a”)); type VI-B CRISPR/Cas effector polypeptides (e.g., Cas13b (also known as C2c4)); and type VI-C CRISPR/Cas effector polypeptides (e.g., Cas13c (also known as C2c7)). To date, class 2 CRISPR/Cas effector polypeptides encompass type II, type V, and type VI CRISPR/Cas effector polypeptides, but the term is also meant to encompass any class 2 CRISPR/Cas effector polypeptide suitable for binding to a corresponding guide RNA and forming an RNP complex.

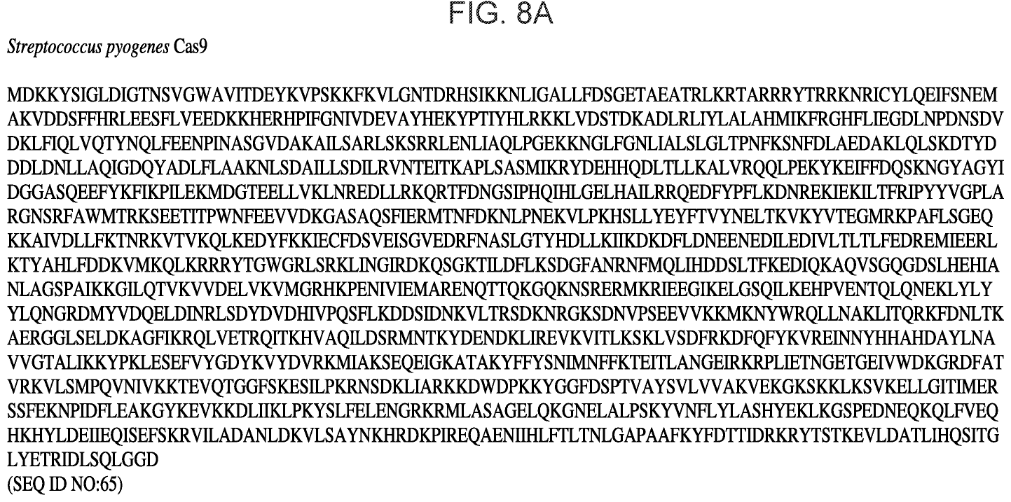

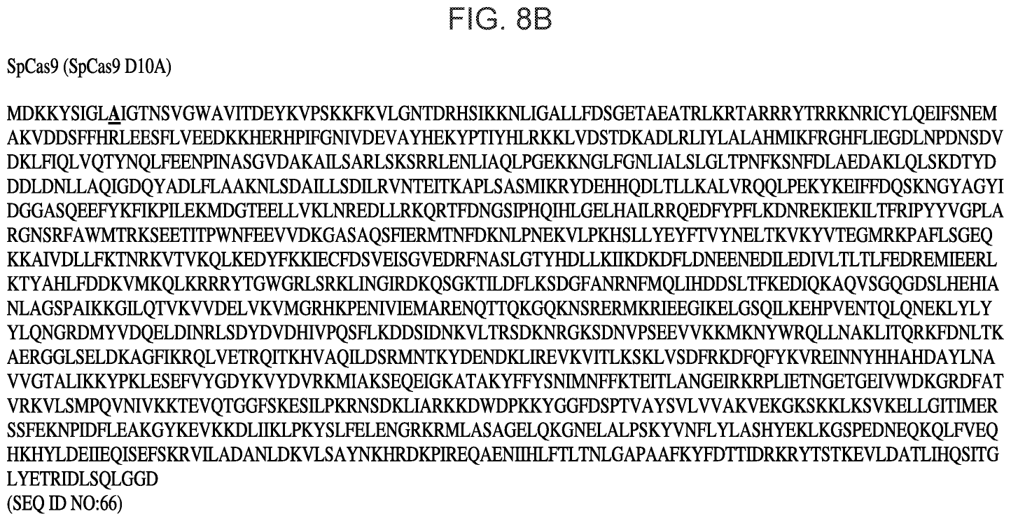

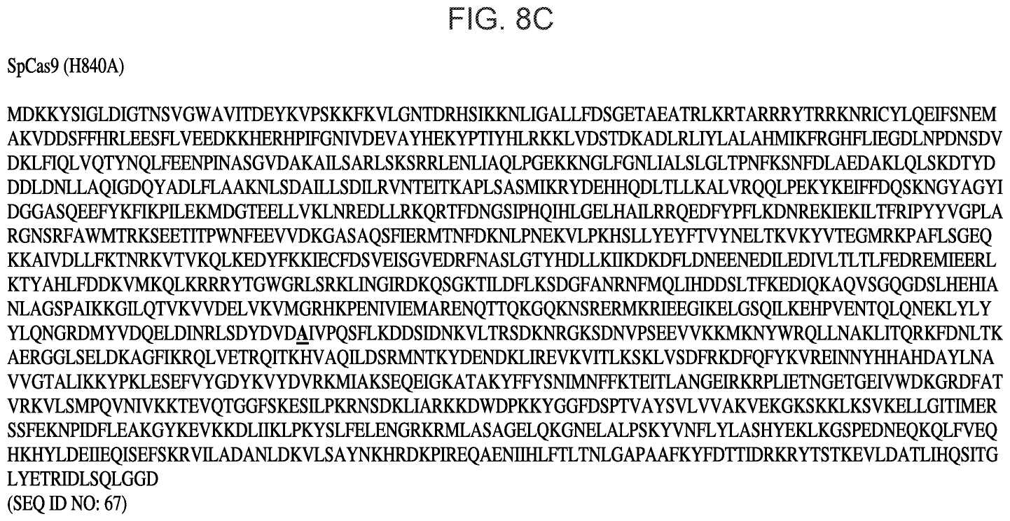

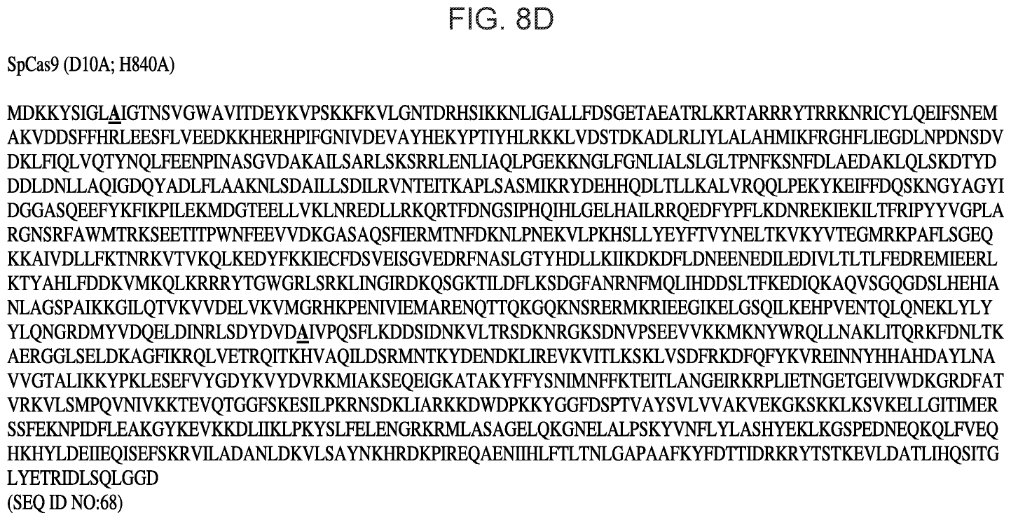

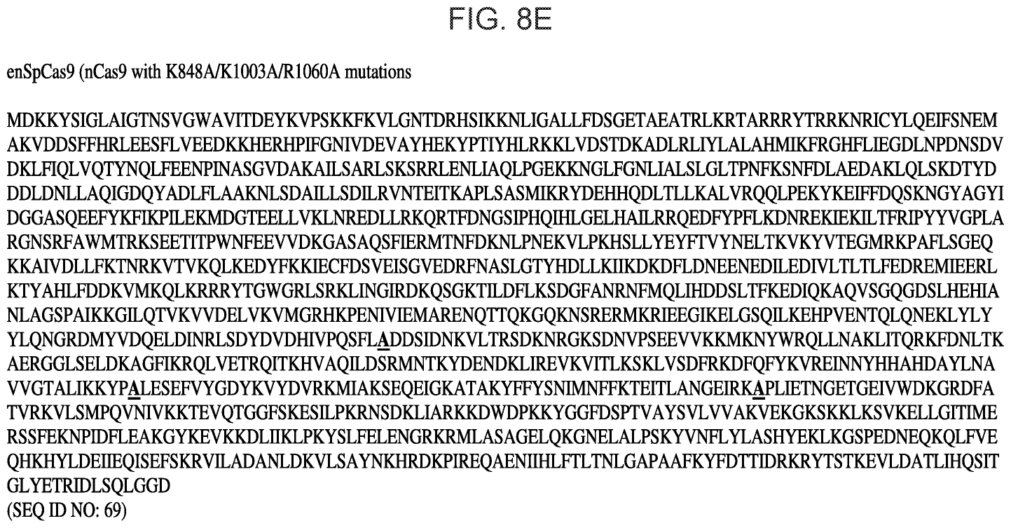

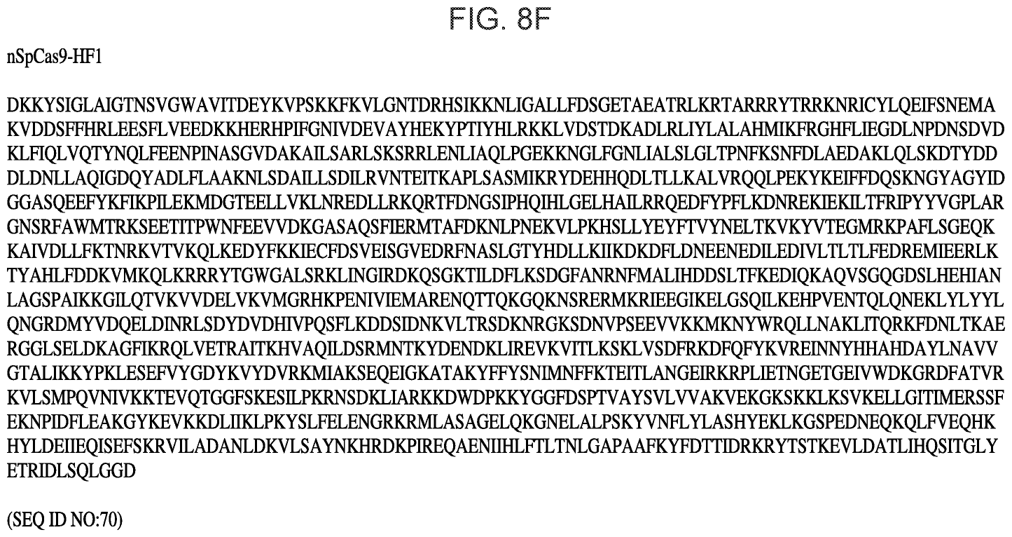

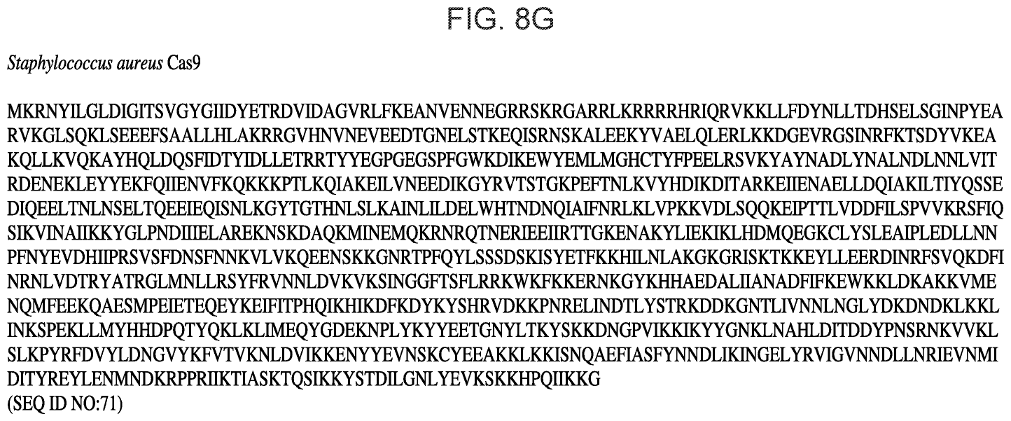

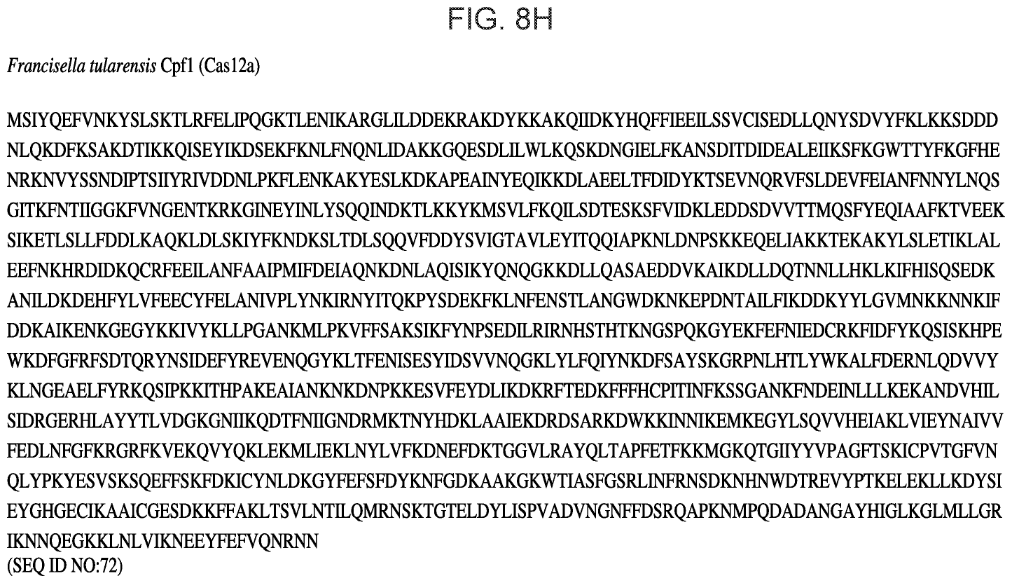

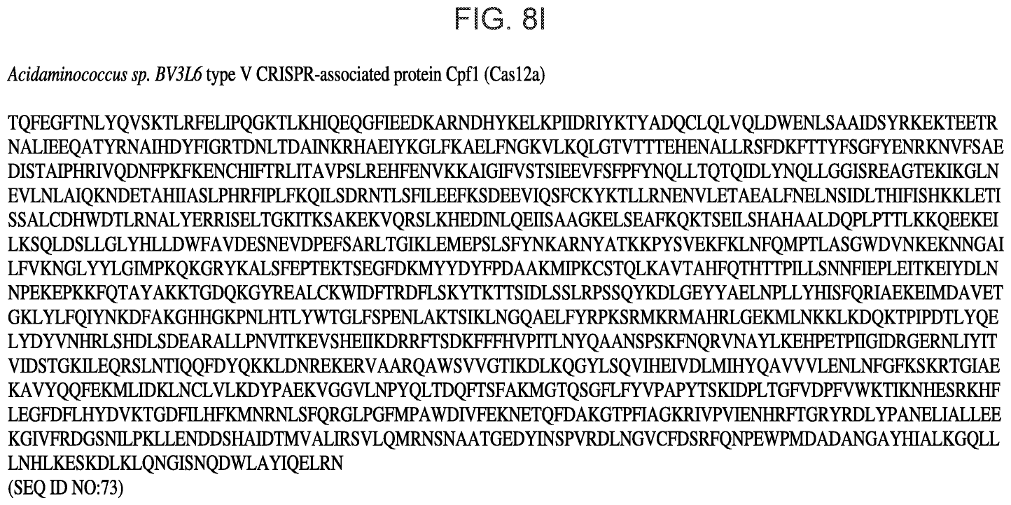

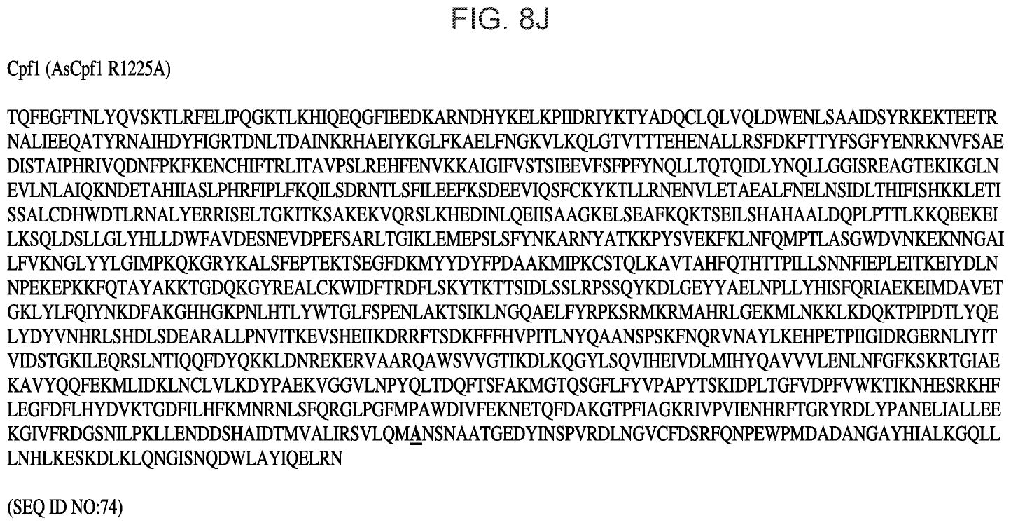

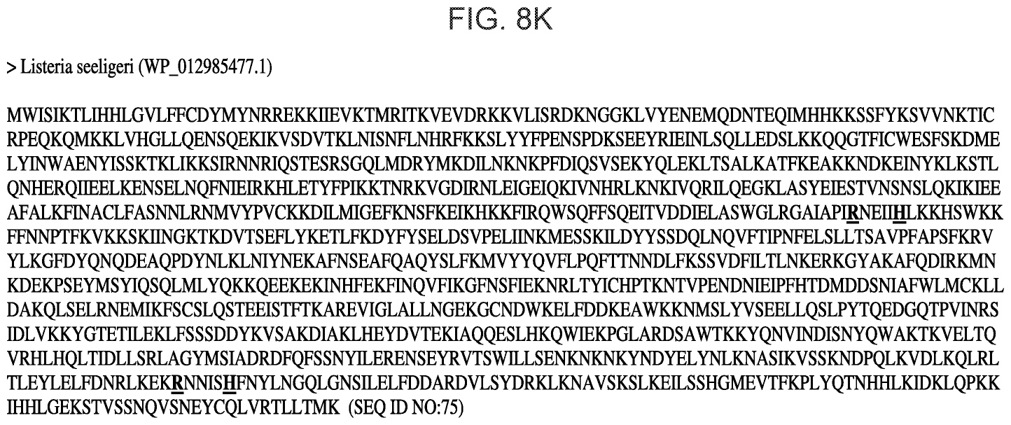

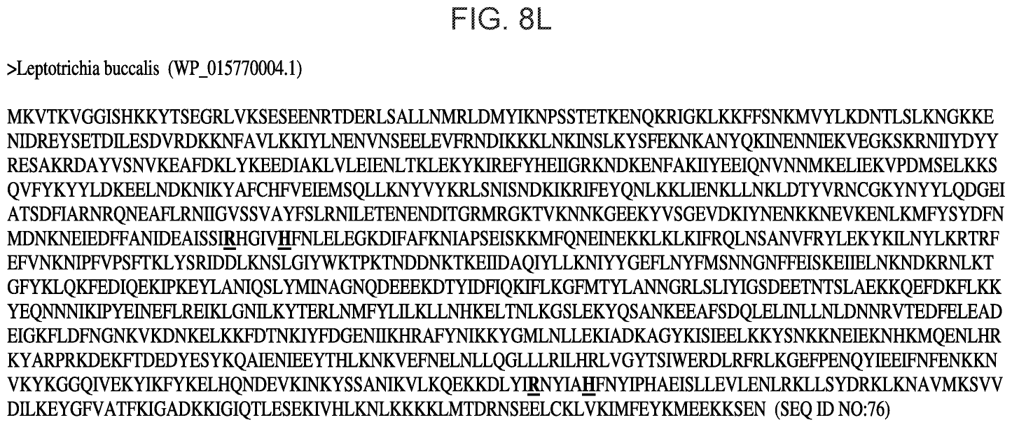

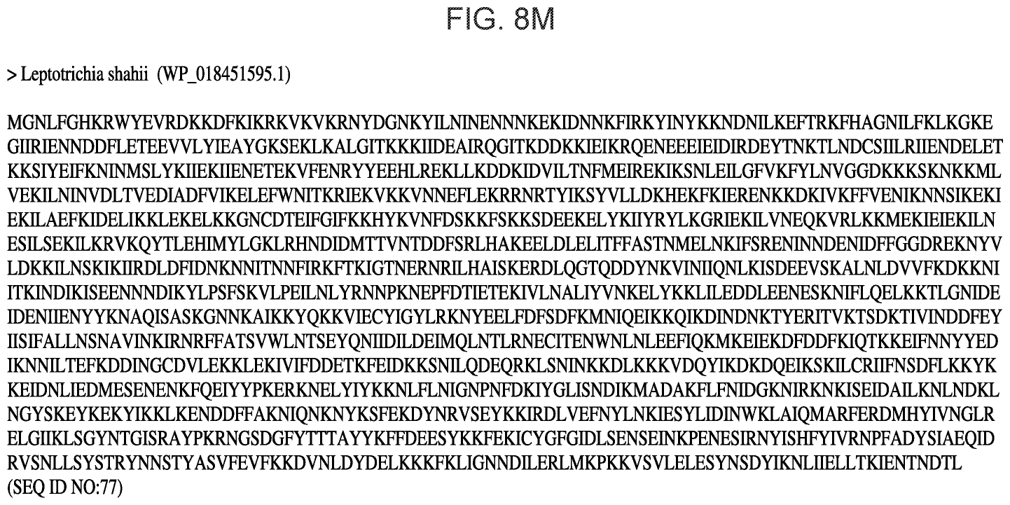

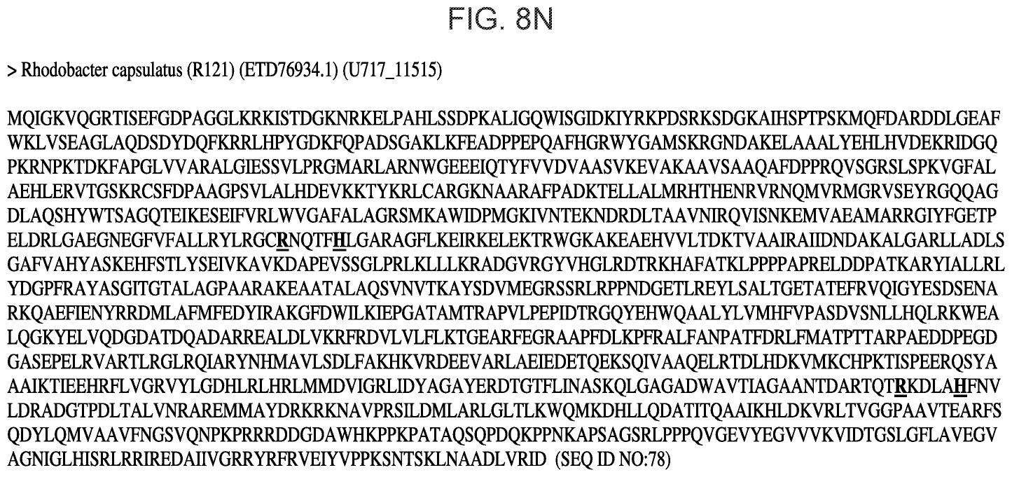

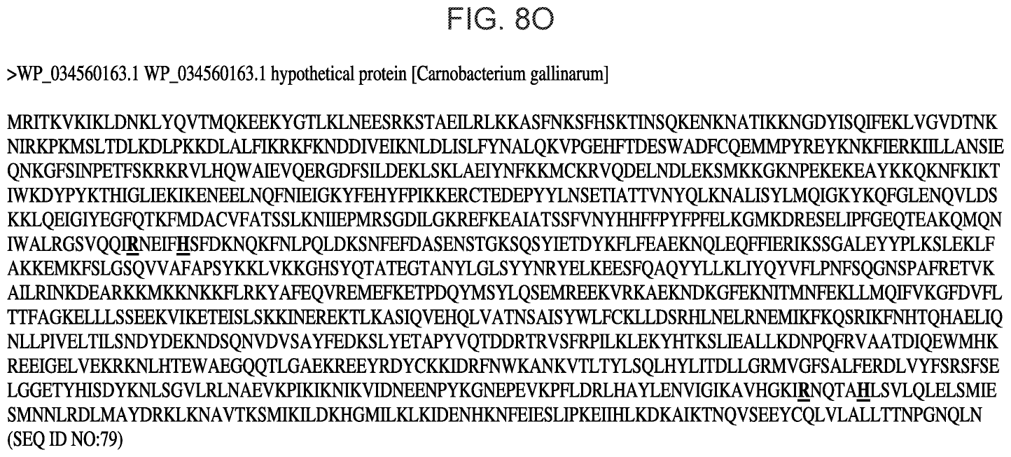

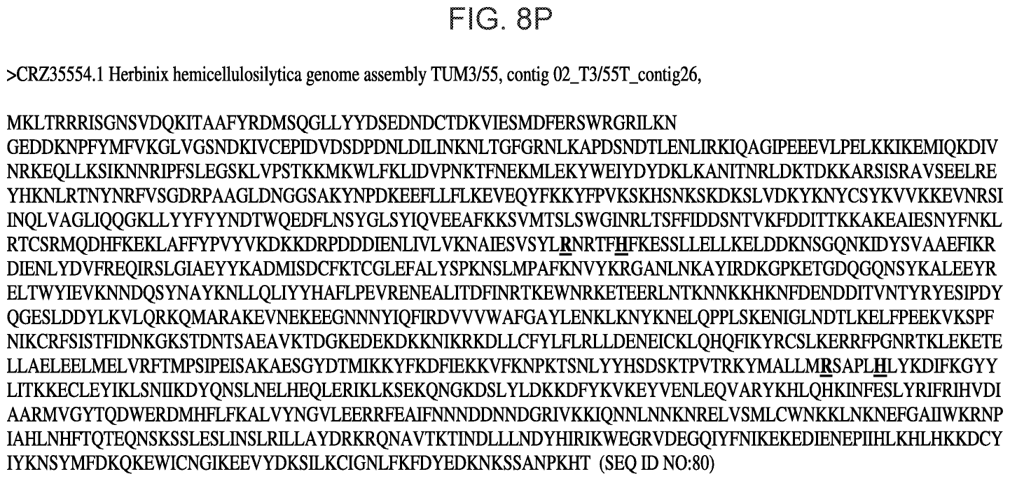

In some cases, the CRISPR/Cas effector polypeptide comprises an amino acid sequence having at least 50%, at least 60%, at least 70%, at least 80%, at least 90%, at least 95%, at least 98%, at least 99%, or 100%, amino acid sequence identity to the amino acid sequence depicted in any one of FIG. 8A-8P.

In some cases, the CRISPR/Cas effector polypeptide is a type II CRISPR/Cas effector polypeptide. In some cases, the CRISPR/Cas effector polypeptide is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 8A. In some cases, the Cas9 polypeptide used in a composition or method of the present disclosure is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 8G.

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 8A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 3A, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, the CRISPR/Cas effector polypeptide is a type V CRISPR/Cas effector polypeptide. In some cases, a type V CRISPR/Cas effector polypeptide is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in any one of FIG. 8H-8J.

A CRISPR/Cas effector polypeptide, when complexed with a guide RNA comprising a nucleotide sequence that is complementary to a target nucleic acid, can provide for: i) replacement of all or a portion of a target nucleic acid (e.g., where a donor nucleic acid is provided); or ii) deletion of all or a portion of a target nucleic acid; or iii) modulation of transcription (e.g., reduction in transcription) of a target nucleic acid (e.g., where the CRISPR/Cas effector polypeptide is a catalytically inactive CRISPR/Cas effector polypeptide fused to a transcription modulatory polypeptide).

### Guide Nucleic Acids

A nucleic acid that binds to a class 2 CRISPR/Cas effector polypeptide (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid. The term “guide RNA”, as used herein, refers to an RNA that comprises: i) an “activator” nucleotide sequence that binds to a CRISPR/Cas effector polypeptide (e.g., a class 2 CRISPR/Cas effector polypeptide such as a type II, type V, or type VI CRISPR/Cas endonuclease) and activates the CRISPR/Cas effector polypeptide; and ii) a “targeter” nucleotide sequence that comprises a nucleotide sequence that hybridizes with a target nucleic acid. The “activator” nucleotide sequence and the “targeter” nucleotide sequence can be on separate RNA molecules (e.g., a “dual-guide RNA”); or can be on the same RNA molecule (a “single-guide RNA”). A guide nucleic acid in some cases includes only ribonucleotides. In some cases, a guide nucleic acid includes both ribonucleotides and deoxyribonucleotides.

Examples and guidance related to type V or type VI CRISPR/Cas endonucleases and guide RNAs (as well as information regarding requirements related to protospacer adjacent motif (PAM) sequences present in targeted nucleic acids) can be found in the art, for example, see Zetsche et al., Cell. 2015 Oct. 22; 163(3):759-71; Makarova et al., Nat Rev Microbiol. 2015 November; 13(11):722-36; and Shmakov et al., Mol Cell. 2015 Nov. 5; 60(3):385-97.

In some cases, a CRISPR/Cas effector polypeptide is an enzymatically inactive CRISPR/Cas effector polypeptide. In some cases, a CRISPR/Cas effector polypeptide is a fusion polypeptide comprising: a) an enzymatically inactive CRISPR/Cas effector polypeptide; and b) a heterologous fusion partner (a heterologous polypeptide). The heterologous polypeptide can be, e.g., a transcription modulator, a nuclease; a base editor; a recombinase; an anti-CRISPR polypeptide; a reverse transcriptase; a prime editor. In some cases, a fusion polypeptide comprises: a) an enzymatically inactive CRISPR/Cas effector polypeptide; and b) a transcription modulator. For example, a fusion polypeptide comprising an enzymatically inactive CRISPR/Cas effector polypeptide and a transcription modulator can be used to reduce transcription of a target nucleic acid.

### Targets of CRISPR-Cas Effector Polypeptide/Guide RNAs

A CRISPR/Cas effector polypeptide, together with a guide RNA, can be targeted to any of a variety of target nucleic acids. For example, suitable targets include nucleic acids encoding polypeptides such as: NF-κB; interferon regulatory factors such as IFN3, IFN7, and the like; MyD88; IFN-β; IFN-γ; transforming growth factor beta receptor-1 (TGFBR1); toll-like receptors; Fc receptors; immune checkpoint inhibitors (e.g., PD1; PDL1; CTLA4; CD47; and the like); and the like. A CRISPR/Cas effector polypeptide, together with a guide RNA, can be targeted to a nucleic acid (e.g., genomic nucleic acid) encoding a polypeptide implicated in Alzheimer Disease, where such polypeptides include, e.g., apolipoprotein E (APOE), apolipoprotein C-1 (APOC1), CD22, CD33, colony stimulating factor 1 receptor (CSF1R), SPP1, tyrosine kinase binding protein (TYROBP), triggering receptor expressed on myeloid cells-2 (TREM2), valosin-containing protein (VCP), ATP binding cassette subfamily D member 1 (ABCD1), protein tyrosine phosphatase receptor type G (PTPRG), cathepsin D (CTSD), brain derived neurotrophic factor (BDNF), arylsulfatase A (ARSA), CX3CR1, and CCR5. In some cases, all or a portion of the target nucleic acid is replaced. In some cases, all or a portion of the target nucleic acid is deleted. In some cases, transcription of the target nucleic acid is modulated (increased or reduced).

Non-limiting examples of target nucleic acids, manipulations, and conditions to be treated, are set out in the table below. (KO: knockout; ko/d: knockout/knockdown)

| Gene (target | | |
|---|---|---|
| nucleic acid) | manipulation | conditions |
| TREM2 | overexpress | Alzheimer disease |
| CSF1R | overexpress or KO, or | aging |
| | point mutation | |
| NFkB | ko | pain, MS |
| CD22 | ko/d | aging |
| PTPRG | ko/d | aging |
| APOC1 | ko/d | aging |
| APOE | ko/d | aging |
| SPP1 | ko/d | aging |
| SPP1 | ko/d | MS |
| CTSD | ko/d | |
| TGFBR1 | overexpress | Alzheimer disease |
| CSF1R | point mutation | hereditary diffuse |
| | | leukoencephalopathy |
| | | with axonal spheroids |
| CCR5 | ko/d or point mutation | HIV associated brain |
| | | injury, HIV-associated |
| | | dementia |
| VCP | point mutation | Alzheimer disease |
| TREM2 | overexpress | multiple sclerosis |
| BDNF | overexpress | |
| TREM2 | point mutation to edit | Alzheimer disease |
| | rs75932628 (encoding | |
| | R47H) | |
| | | Tauopathy? |
| BIN1 | | Late Onset Alzheimer |
| | | Disease |
| CD33 | k/d | Alzheimer disease |
| ABCD1 | gene/enzyme | X-linked |
| | replacement | adrenoleukodystrophy |
| | | (ALD) |
| ARSA | gene/enzyme | metachromatic |
| | replacement | leukodystrophy (MLD) |
| | gene/enzyme | |
| | replacement | |
| TYROBP | inactivate | PLOSL |
| TYROBP | inactivate | Alzheimer disease |
| CCR2 | inactivate | viral encephalitis |
| CX3CR1 | inactivate | viral encephalitis |

### Nucleic Acid Gene Products

Suitable nucleic acid gene products include interfering RNA, antisense RNA, ribozymes, and aptamers. Where the gene product is an interfering RNA (RNAi), suitable RNAi include RNAi that decrease the level of a disease-related protein in a cell. For example, an RNAi can be a miRNA, an shRNA, or an siRNA that reduces the level of a pro-inflammatory polypeptide in a macrophage, e.g., a microglial cell. A nucleic acid gene product can also be a CRISPR/Cas guide RNA.

### Regulatory Elements

In some cases, a nucleotide sequence encoding a heterologous gene product of interest is operably linked to a transcriptional control element. For example, in some cases, a nucleotide sequence encoding a heterologous gene product of interest is operably linked to a constitutive promoter. In other cases, a nucleotide sequence encoding a heterologous gene product of interest is operably linked to an inducible promoter. In some instances, a nucleotide sequence encoding a heterologous gene product of interest is operably linked to a tissue-specific or cell type-specific regulatory element. For example, in some instances, a nucleotide sequence encoding a heterologous gene product of interest is operably linked to a macrophage-specific promoter. In some cases, a nucleotide sequence encoding a heterologous gene product of interest is operably linked to a microglial cell-specific promoter.

### Pharmaceutical Compositions

The present disclosure provides a pharmaceutical composition comprising: a) a subject rAAV virion, as described above; and b) a pharmaceutically acceptable carrier, diluent, excipient, or buffer. In some cases, the pharmaceutically acceptable carrier, diluent, excipient, or buffer is suitable for use in a human.

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

### Methods

### Methods of Delivering a Gene Product

The present disclosure provides a method of delivering one or more gene products to a macrophage in an individual, the method comprising administering to the individual a subject rAAV virion as described above. The one or more gene products can be a polypeptide (e.g., an antibody specific for a pro-inflammatory polypeptide; a CAR; an immune checkpoint inhibitor; and the like), an interfering RNA (e.g., an shRNA, an siRNA, and the like), an aptamer, a gene-editing polypeptide (e.g., a CRISPR-Cas effector polypeptide), or a CRISPR/Cas guide RNA, etc., as described above. Delivering a gene product to a macrophage can provide for treatment of various diseases and disorders, including inflammatory diseases, neurological diseases, and cancer. In some cases, the macrophage is a microglial cell.

The present disclosure provides a method modifying a target nucleic acid in a macrophage, the method comprising contacting the macrophage with: 1) an rAAV virion of the present disclosure, wherein the rAAV virion comprises a heterologous nucleic acid comprising a nucleotide sequence encoding a CRISPR/Cas effector polypeptide that binds a guide RNA; and 2) the guide RNA. The present disclosure provides a method modifying a target nucleic acid in a macrophage, the method comprising contacting the macrophage with an rAAV virion of the present disclosure, wherein the rAAV virion comprises a heterologous nucleic acid comprising a nucleotide sequence encoding: i) a CRISPR/Cas effector polypeptide that binds a guide RNA; and ii) the guide RNA. In some cases, the method comprises contacting the macrophage with a donor DNA template. In some cases, the CRISPR/Cas effector polypeptide is a Cas9 polypeptide. In some cases, the guide RNA is a single-guide RNA.

The present disclosure provides a method of delivering a gene product to a macrophage in an individual, the method comprising administering to the individual a subject rAAV virion as described above. The gene product can be a polypeptide or an interfering RNA (e.g., an shRNA, an siRNA, and the like), an aptamer, or a gene-editing polypeptide (e.g., a CRISPR/Cas effector polypeptide), as described above. Delivering a gene product to a macrophage can provide for treatment of an inflammatory disease or a cancer.

### Treatment Methods

The present disclosure provides methods of treatment of an individual in need thereof, where the methods generally involve administering to the individual an effective amount of an rAAV virion of the present disclosure. A method of the present disclosure can be used to treat a neurodegenerative disease; spinal cord injury (SCI); traumatic brain injury (TBI); cancers, e.g., CNS tumors, glioblastoma multiforme, and the like; human immunodeficiency virus (HIV) infection; and leukodystrophies.

The present disclosure provides a method of treating a disease or condition (e.g., a neurodegenerative disease or disorder; TBI; SCI; or a brain cancer), the method comprising administering to an individual in need thereof an effective amount of a subject rAAV virion as described above. A subject rAAV virion can be administered via intracranial injection, or by any other convenient mode or route of administration. Other convenient modes or routes of administration include, e.g., intracerebroventicular, intrathecal, intra-cisterna magna, or intravenous etc.

A “therapeutically effective amount” will fall in a relatively broad range that can be determined through experimentation and/or clinical trials. For example, for in vivo injection, i.e., injection directly into the brain, a therapeutically effective dose will be on the order of from about 106 to about 1015 of the rAAV virions, e.g., from about 108 to 1012 rAAV virions. For in vitro transduction, an effective amount of rAAV virions to be delivered to cells will be on the order of from about 108 to about 1013 of the rAAV virions. Other effective dosages can be readily established by one of ordinary skill in the art through routine trials establishing dose response curves.

In some cases, more than one administration (e.g., two, three, four or more administrations) may be employed to achieve the desired level of gene expression. In some cases, the more than one administration is administered at various intervals, e.g., daily, weekly, twice monthly, monthly, every 3 months, every 6 months, yearly, etc. In some cases, multiple administrations are administered over a period of time of from 1 month to 2 months, from 2 months to 4 months, from 4 months to 8 months, from 8 months to 12 months, from 1 year to 2 years, from 2 years to 5 years, or more than 5 years.

### Methods of Treating a Neurodegenerative Disease

Neurodegenerative diseases and disorders that can be treated using a subject method include neurodegenerative diseases and disorders of the central nervous system (CNS). Neurodegenerative diseases and disorders that can be treated using a subject method include, but are not limited to, multiple sclerosis (MS), Lewy Body dementia, Alzheimer disease, Parkinson's disease, Huntington's disease, amyotrophic lateral sclerosis, spinocerebellar ataxia, Down Syndrome, and the like. As a non-limiting example, an rAAV virion can be used to deliver one or more polypeptides that provide for an anti-inflammatory effect, e.g., an anti-TNFα antibody, as described above, where production of the anti-inflammatory polypeptide(s) by a microglial cell provides for treatment of the neurodegenerative disease or disorder.

One of ordinary skill in the art can readily determine an effective amount of an rAAV virion by testing for an effect of administration of an rAAV virion of the present disclosure on one or more parameters, such as a symptom associated with a neurodegenerative disease. In some cases, administering an effective amount of an rAAV virion of the present disclosure results in a decrease in the rate of loss of brain function, anatomical integrity of the brain, or brain health, e.g. a 2-fold, 3-fold, 4-fold, or 5-fold or more decrease in the rate of loss and hence progression of disease, e.g. a 10-fold decrease or more in the rate of loss and hence progression of disease. In some cases, administering an effective amount of an rAAV virion of the present disclosure results in a gain in brain function, an improvement in brain anatomy or health, and/or a stabilization in brain function, e.g. a 2-fold, 3-fold, 4-fold or 5-fold improvement or more in brain function, brain anatomy or health, e.g. a 10-fold improvement or more in brain function, brain anatomy or health, and/or stability of the brain. Brain function can include, e.g., cognitive function.

### Methods of Treating TBI and SCI

A method of the present disclosure can be used to treat TBI. A method of the present disclosure can be used to treat SCI. One of ordinary skill in the art can readily determine an effective amount of an rAAV virion for treating TBI by testing for an effect of administration of an rAAV virion of the present disclosure on one or more parameters, such as brain function, anatomical integrity of the brain, or brain health. One of ordinary skill in the art can readily determine an effective amount of an rAAV virion for treating SCI injury by testing for an effect of administration of an rAAV virion of the present disclosure on one or more parameters, such as motor function.

### Methods of Treating Cancer

As noted above, a method of the present disclosure can be used to treat a cancer. For example, an rAAV virion of the present disclosure can be used to deliver to a microglial cell one or more polypeptides that provide an anti-tumor effect. Such polypeptides can include, e.g., a CAR, an immune checkpoint inhibitor, and the like, as described above. The present disclosure provides a method of treating a cancer in an individual, the method comprising administering to the individual an effective amount of an rAAV virion of the present disclosure. In some cases, an “effective amount” of an rAAV virion of the present disclosure is an amount that, when administered in one or more doses to an individual in need thereof, reduces the number of cancer cells in the individual. For example, in some cases, an “effective amount” of an rAAV virion of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the rAAV virion, or in the absence of administration with the rAAV virion. In some cases, an “effective amount” of an rAAV virion of the present disclosure is an amount that, when administered in one or more doses to an individual in need thereof, reduces the number of cancer cells in the individual to undetectable levels.

In some cases, an “effective amount” of an rAAV virion of the present disclosure is an amount that, when administered in one or more doses to an individual in need thereof, reduces the tumor mass in the individual. For example, in some cases, an “effective amount” of an rAAV virion of the present disclosure is an amount that, when administered in one or more doses to an individual in need thereof (an individual having a tumor), reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the rAAV virion, or in the absence of administration with the rAAV virion. In some cases, an “effective amount” of an rAAV virion of the present disclosure is an amount that, when administered in one or more doses to an individual in need thereof (an individual having a tumor), reduces the tumor volume in the individual. For example, in some cases, an “effective amount” of an rAAV virion of the present disclosure is an amount that, when administered in one or more doses to an individual in need thereof (an individual having a tumor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the rAAV virion, or in the absence of administration with the rAAV virion. In some cases, an “effective amount” of an rAAV virion of the present disclosure is an amount that, when administered in one or more doses to an individual in need thereof, increases survival time of the individual. For example, in some cases, an “effective amount” of an rAAV virion of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the rAAV virion.

Cancers that can be treated using a method of the present disclosure include, e.g., brain cancers such as gliomas, including astrocytomas, ependymomas, glioblastomas, medulloblastomas, and oligodendrogliomas. Cancers that can be treated using a method of the present disclosure include glioblastoma multiforme. Brain cancers that can be treated using a method of the present disclosure include primary brain cancers and metastatic brain cancers.

### Methods of Treating a Leukodystrophy

A method of the present disclosure can be used to treat a leukodystrophy. Leukodystrophies include, e.g., adult-onset autosomal dominant leukodystrophy, Aicardi-Goutieres syndrome, Alexander disease, CADASIL, Canavan disease, CARASIL, cerebrotendinous xanthomatosis, childhood ataxia and cerebral hypomyelination/vanishing white matter disease, Fabry disease, fucosidosis, GM1 gangliosidosis, Krabbe disease, L-2-hydroxyglutaric aciduria, megalencephalic leukoencephalopathy with subcortical cysts, metachromatic leukodystrophy (MLD), multiple sulfatase deficiency, Pelizaeus-Merzbacher disease, PolIII-related leukodystrophies, Refsum disease, salla disease (free sialic acid storage disease), Sjorgren-Larsson syndrome, X-linked adrenoleukodystrophy (ALD), hereditary diffuse leukoencephalopathy with axonal spheroids, polycystic lipomembranous osteodyplasia with sclerosing leukoencephalopathy (PLOSL), and Zellweger syndrome spectrum disorders.

### Methods of Treating HIV Infection

A method of the present disclosure for treating an HIV infection in an individual can involve delivering a CRISPR/Cas effector polypeptide and a guide RNA targeting CCR5 to microglia. Thus, the heterologous nucleic acid present in the rAAV virion can comprise nucleotide sequences encoding a CRISPR/Cas effector polypeptide and a guide RNA targeting CCR5. The CCR5 gene in the microglia can be knocked out such that the microglia exhibit reduced cell surface expression of CCR5, or undetectable cell surface expression of CCR5.

### Nucleic Acids and Host Cells

The present disclosure provides an isolated nucleic acid comprising a nucleotide sequence that encodes a subject variant adeno-associated virus (AAV) capsid protein as described above.

A subject recombinant AAV vector can be used to generate a subject recombinant AAV virion, as described above. Thus, the present disclosure provides a recombinant AAV vector that, when introduced into a suitable cell, can provide for production of a subject recombinant AAV virion.

The present disclosure further provides host cells, e.g., isolated (genetically modified) host cells, comprising a subject nucleic acid. A subject host cell can be an isolated cell, e.g., a cell in in vitro culture. A subject host cell is useful for producing a subject rAAV virion, as described below. Where a subject host cell is used to produce a subject rAAV virion, it is referred to as a “packaging cell.” In some cases, a subject host cell is stably genetically modified with a subject nucleic acid. In other cases, a subject host cell is transiently genetically modified with a subject nucleic acid.

A subject nucleic acid is introduced stably or transiently into a host cell, using established techniques, including, but not limited to, electroporation, calcium phosphate precipitation, liposome-mediated transfection, and the like. For stable transformation, a subject nucleic acid will generally further include a selectable marker, e.g., any of several well-known selectable markers such as neomycin resistance, and the like.

A subject host cell is generated by introducing a subject nucleic acid into any of a variety of cells, e.g., mammalian cells, including, e.g., murine cells, and primate cells (e.g., human cells). Suitable mammalian cells include, but are not limited to, primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like. A subject host cell can also be made using a baculovirus to infect insect cells such as Sf9 cells, which produce AAV (see, e.g., U.S. Pat. No. 7,271,002; U.S. patent application Ser. No. 12/297,958).

In some cases, a subject genetically modified host cell includes, in addition to a nucleic acid comprising a nucleotide sequence encoding a variant AAV capsid protein, as described above, a nucleic acid that comprises a nucleotide sequence encoding one or more AAV rep proteins. In other cases, a subject host cell further comprises an rAAV vector. An rAAV virion can be generated using a subject host cell. Methods of generating an rAAV virion are described in, e.g., U.S. Patent Publication No. 2005/0053922 and U.S. Patent Publication No. 2009/0202490.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A recombinant adeno-associated virus (rAAV) virion comprising:
a1) a variant AAV capsid protein, wherein the variant AAV capsid protein comprises an amino acid sequence having at least 95% amino acid sequence identity to the amino acid sequence depicted in FIG. 6A, wherein the amino acid at position 467 is other than Gly, the amino acid at position 551 is other than Ala, the amino acid at position 665 is other than Pro, and the amino acid at position 719 is other than Glu; and
b1) a heterologous nucleic acid comprising one or more nucleotide sequences encoding one or more heterologous gene products; or
a2) a variant AAV capsid protein, wherein the variant AAV capsid protein comprises an amino acid sequence having at least 95% amino acid sequence identity to the amino acid sequence depicted in any one of FIG. 9A-9M; and
b2) a heterologous nucleic acid comprising one or more nucleotide sequences encoding one or more heterologous gene products.

Aspect 2. The rAAV virion of aspect 1, wherein the amino acid at position 467 is Ala, the amino acid at position 551 is Lys, the amino acid at position 665 is Ala, and the amino acid at position 719 is Asp.

Aspect 3. The rAAV virion of aspect 1, wherein the amino acid at position 264 is a T, Q, or A, position 448 is an S or A, position 459 is a T or N, position 470 is an S or A, position 495 is an S or T, position 533 is a D or E, position 547 is a Q, E, or T, position 555 is a T or A, position 557 is an E or D, position 561 is an M, L, or I, position 563 is an S or N, position 593 is an A, Q, or V, position 596 is an A or T, position 661 is an A, E, or T, position 662 is a V, T, or A, position 664 is a T or S, position 718 is an N or S, and position 723 is an S or T.

Aspect 4. The rAAV virion of aspect 1 or aspect 2, wherein:
i) the amino acids at positions 264, 448, 459, 470, 495, 533, 547, 555, 557, 561, 563, 593, 596, 661, 662, 664, 718 and 723 are A, A, N, S, S, D, E, A, E, L, N, A, A, A, T, T, N, and S, respectively; or
ii) the amino acids at positions 264, 448, 459, 470, 495, 533, 547, 555, 557, 561, 563, 593, 596, 661, 662, 664, 718 and 723 are Q, S, N, A, S, E, Q, T, D, M, S, Q, T, A, V, S, S, and S, respectively; or
iii) the amino acids at positions 264, 448, 459, 470, 495, 533, 547, 555, 557, 561, 563, 593, 596, 661, 662, 664, 718 and 723 are A, A, T, S, T, D, Q, A, D, I, N, A, T, T, V, S, S, and T, respectively.

Aspect 5. The rAAV virion of aspect 1, wherein the amino acids at positions 264, 448, 459, 470, 495, 533, 547, 555, 557, 561, 563, 593, 596, 661, 662, 664, 718 and 723 are A, S, N, S, S, D, Q, A, E, M, S, Q, A, A, V, T, S, and S, respectively.

Aspect 6. The rAAV virion of aspect 1, wherein the variant capsid polypeptide comprises the amino acid sequence depicted in any one of FIG. 6B and FIG. 9A-9M.

Aspect 7. The rAAV virion of aspect 1, wherein the variant capsid polypeptide comprises the amino acid sequence depicted in FIG. 6B.

Aspect 8. The rAAV virion of any one of aspects 1-7, wherein the rAAV virion exhibits at least 5-fold increased infectivity of a macrophage compared to the infectivity of the macrophage by a control AAV virion comprising the corresponding parental AAV capsid protein, optionally wherein the macrophage is a microglial cell.

Aspect 9. The rAAV virion of any one of aspects 1-8, wherein the one or more heterologous gene products comprises an interfering RNA or an aptamer.

Aspect 10. The rAAV virion of any one of aspects 1-8, wherein the wherein the one or more heterologous gene products comprises a polypeptide.

Aspect 11. The rAAV virion of aspect 10, wherein the polypeptide is an anti-inflammatory polypeptide, an immunosuppressive polypeptide, an immune checkpoint inhibitor, or a chimeric antigen receptor.

Aspect 12. The rAAV virion of aspect 10, wherein the polypeptide is a chimeric antigen receipt, a cytokine, an antibody, a T-cell receptor, an NF-κB pathway polypeptide, an interferon signaling pathway polypeptide, an immune checkpoint inhibitor, or a transcription factor.

Aspect 13. The rAAV virion of aspect 10, wherein the polypeptide generates a detectable signal.

Aspect 14. The rAAV virion of aspect 13, wherein the polypeptide is a luciferase or a fluorescent polypeptide.

Aspect 15. The rAAV virion of aspect 10, wherein the polypeptide is a genome-editing enzyme.

Aspect 16. The rAAV virion of aspect 15, wherein the genome-editing enzyme is a CRISPR/Cas effector polypeptide, a zinc finger nuclease, or a TALEN.

Aspect 17. The rAAV virion of aspect 10, wherein the polypeptide is a CRISPR/Cas effector polypeptide.

Aspect 18. The rAAV virion of aspect 17, wherein the CRISPR/Cas effector polypeptide is a type II CRISPR/Cas effector polypeptide, a type V CRISPR/Cas effector polypeptide, or a type VI CRISPR/Cas effector polypeptide.

Aspect 19. The rAAV virion of any one of aspects 1-8, wherein the one or more heterologous gene products comprise a CRISPR/Cas effector polypeptide and a guide RNA.

Aspect 20. The rAAV virion of aspect 19, wherein the guide RNA comprises a nucleotide sequence that targets a polypeptide selected from an NF-κB pathway polypeptide, an interferon signaling pathway polypeptide, apolipoprotein E (APOE), apolipoprotein C-1 (APOC1), CD22, colony stimulating factor 1 receptor (CSFIR), SPP1, tyrosine kinase binding protein (TYROBP), triggering receptor expressed on myeloid cells-2 (TREM2), valosin-containing protein (VCP), and CCR5.

Aspect 21. A pharmaceutical composition comprising:
a) a recombinant adeno-associated virus virion of any one of aspects 1-20; and
b) a pharmaceutically acceptable excipient.

Aspect 22. A method of delivering a gene product to a macrophage in an individual, the method comprising administering to the individual a recombinant adeno-associated virus (rAAV) virion according any one of aspects 1-20 or the composition of aspect 21, optionally wherein the macrophage is a microglial cell.

Aspect 23. The method of aspect 22, wherein said administering is by intracranial, intracerebroventicular, intrathecal, intra-cisterna magna, or intravenous injection.

Aspect 24. A method of treating a neurological disease or disorder in an individual, the method comprising administering to the individual in need thereof an effective amount of a recombinant adeno-associated virus (rAAV) virion according to any one of aspects 1-20 or the composition of aspect 21.

Aspect 25. The method of aspect 24, wherein the neurological disease or disorder is Alzheimer disease, Parkinson's disease, Huntington's disease, multiple sclerosis, or Down syndrome.

Aspect 26. A method of treating a cancer in an individual, the method comprising administering to an individual in need thereof an effective amount of a recombinant adeno-associated virus (rAAV) virion according to any one of aspects 1-20 or the composition of aspect 21.

Aspect 27. The method of aspect 26, wherein the cancer is a glioma.

Aspect 28. The method of aspect 27, wherein the cancer is a glioblastoma.

Aspect 29. An isolated nucleic acid comprising a nucleotide sequence that encodes a variant adeno-associated virus (AAV) capsid protein, wherein the variant AAV capsid protein comprises
a) an amino acid sequence having at least 95% amino acid sequence identity to the amino acid sequence depicted in FIG. 6A, wherein the amino acid at position 467 is other than Gly, the amino acid at position 551 is other than Ala, the amino acid at position 665 is other than Pro, and the amino acid at position 719 is other than Glu; or
b) a variant AAV capsid protein, wherein the variant AAV capsid protein comprises an amino acid sequence having at least 95% amino acid sequence identity to the amino acid sequence depicted in any one of FIG. 9A-9M.

Aspect 30. An isolated, genetically modified host cell comprising the nucleic acid of aspect 29.

Aspect 31. A variant adeno-associated virus (AAV) capsid protein comprising:
a) an amino acid sequence having at least 95% amino acid sequence identity to the amino acid sequence depicted in FIG. 6A, wherein the amino acid at position 467 is other than Gly, the amino acid at position 551 is other than Ala, the amino acid at position 665 is other than Pro, and the amino acid at position 719 is other than Glu; or
b) a variant AAV capsid protein, wherein the variant AAV capsid protein comprises an amino acid sequence having at least 95% amino acid sequence identity to the amino acid sequence depicted in any one of FIG. 9A-9M.

Aspect 32. The variant AAV capsid protein of aspect 31, wherein the amino acid at position 467 is Ala, the amino acid at position 551 is Lys, the amino acid at position 665 is Ala, and the amino acid at position 719 is Asp.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

AAV libraries comprising variant capsid proteins were screened for the ability to infect brain microglial cells. For library design, multiple techniques were applied. These included error-prone polymerase chain reaction (PCR), random 7-mer peptide insertion, structure-guided SCHEMA recombination, and ancestral reconstruction. These techniques were applied to genetically diversify the cap gene and develop variants that can potentially overcome current delivery barriers. Several libraries were used for this screen: 1) a library based on an ancestral AAV sequence and a 7mer peptide display library at position amino acid ˜591 with 5′ TG linker and a 3′ GLS linker (Santiago-Ortiz et al. (2015) Gene Ther. 22:934); 2) a 7mer peptide display library based on AAV1 and AAV2 containing a 7mer peptide insertion at amino acid ˜588, and surrounded by a 5′ LA linker and a 3′ A linker; 3) a 7mer peptide display library based on AAV5 with a 7mer peptide insertion at amino acid ˜575 with 5′ TG linker and a 3′ GLS linker; 4) a shuffled library composed of cap genes from AAV1, 2, 4, 5, 6, 8 and 9 (Koerber et al. (2008) Mol. Ther. 16:1703); 5) a library based on AAV1-4 and 6-9 shuffled region and subjected to further mutagenesis; 6) an AAV5-based library with random mutations; 7) a computer-assisted SCHEMA library comprised of AAV2, 4, 5, 6, 8, and 9 (Ojala et al. (2018) Mol. Ther. 26:304); and 8) a library based on an ancestral AAV sequence (with no peptide inserts) (Santiago-Ortiz et al. (2015) Gene Ther. 22:934).

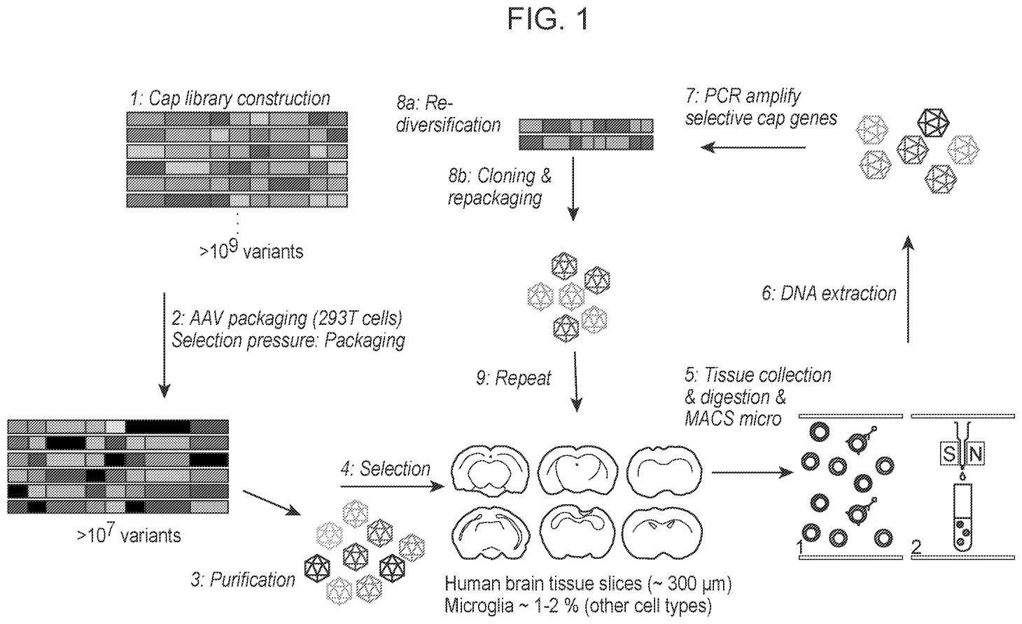

Each library was individually packaged such that each viral genome was encapsulated within the capsid protein shell that is genome encoded. Therefore, functional improvements identified through selection can be linked to the genome sequence contained within the viral capsid. Specifically, the aforementioned nine libraries were transfected separately into packaging cell lines (HEK 293T cells) to produce viral particles. After purification of viral particles and titer quantifications for each individual library using real-time qPCR, all of the libraries were mixed using an equimolar ratio as the initial AAV library pool. From this combination of diverse libraries, an iterative in vitro screening selection process was applied to identify variants with the ability to infect the microglia cells in primary human brain tissues, as depicted schematically in FIG. 1.

Primary brain samples were harvested from a prenatal live brain ranging from 19 to 23 week-old, and sectioned into 300 um thickness. Approximately 50 μL of 10E12-10E13 vg/mL titer library virus was applied directly onto the slice and infected tissues were harvested after 72 hrs of infection. The combined library was administered directly onto each slice at a multiplicity of infection (MOI)=100K, which was calculated based on an estimation of microglia cell number within each brain slice. Upon harvest, brain slices were enzymatically dissociated into single-cell suspension. Functional selective pressure was imposed by harvesting only the microglia population using magnetic-activated cell sorting (MACS). Such method yields ˜95% microglia-only population after sorting. Using Hirt-extraction protocol and PCR-based recovery of cap variants, cap genes of viral genome that were successfully entered microglia cells only after each round were selectively amplified. Recovered cap genes were then used for subsequent AAV cloning and packaging, which eventually drive convergence toward the fittest clones that transduce human microglia cells.

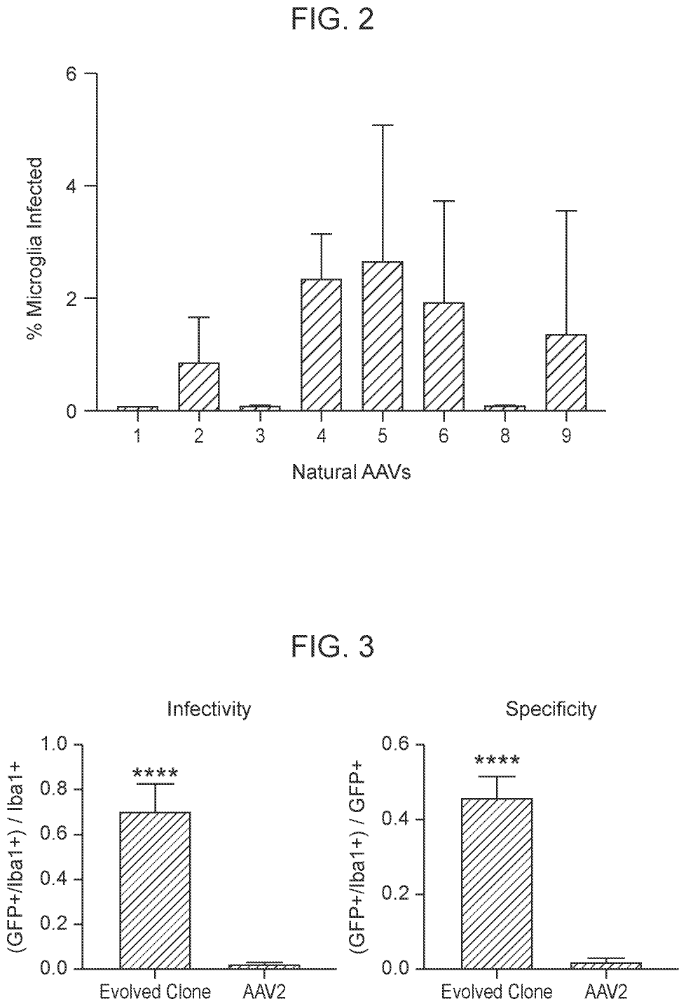

Natural AAV serotypes (i.e. AAV1 to 9) transduce primary human brain tissue poorly and exhibit lack of microglial targeting ability. A nucleic acid encoding green fluorescent protein (GFP) was packaged within each type of natural existing AAV serotypes (AAV1-9). As shown in FIG. 2, that few (usually fewer than 5%) of the GFP transgene—expressing cells were microglia (visualized using immunohistochemistry for Iba1, a marker of microglia) in all recombinant natural AAV serotypes. Furthermore, the total number of GFP+ cells was low as well, indicating inefficient transduction of existing vectors on human brain tissues.

Following three rounds of selection, Sanger sequencing analysis of 30 clones was used to evaluate convergence of variants that increased over the rounds in the AAV library. The analysis revealed a convergence of one fittest clone from the ancestral library represented ˜53% of the clones recovered. Out of a library of ˜10E+6-10E+7 unique variants per library, an increase of representation in the viral library indicates positive selection and ability to infect the microglia cells from the human brain tissues.

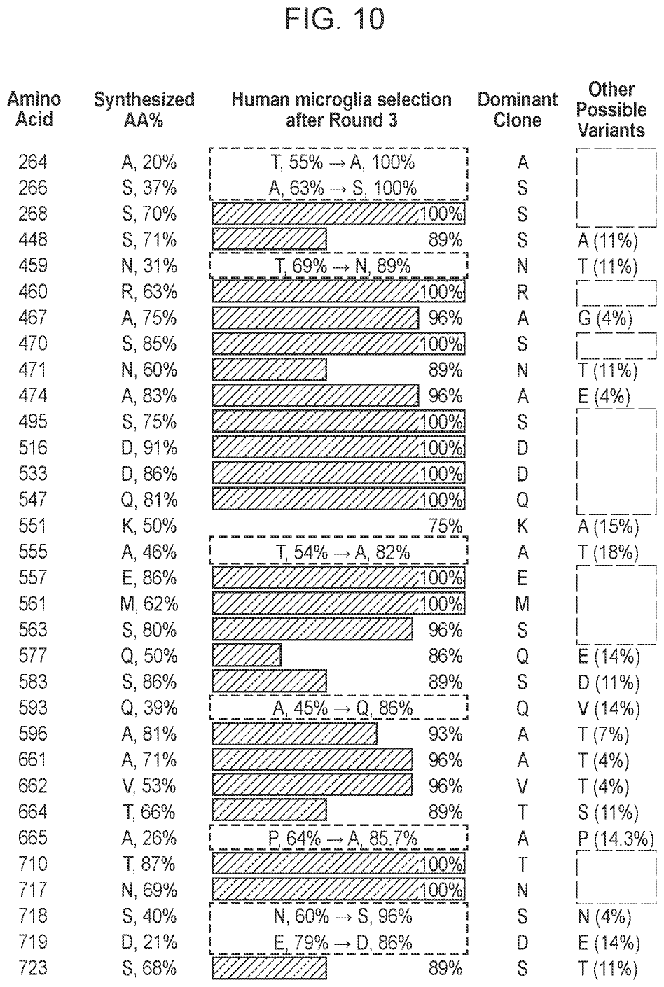

FIG. 6B provides the amino acid sequence of a dominant clone. FIG. 9A-9M provide amino acid sequences of additional variants. FIG. 10 depicts the calculated percent of dominant amino acids at each of 32 variable positions in AAV capsid proteins after three rounds of selection.

To validate if the evolved clone can transduce microglia cells more efficiently and specifically, the fittest clone cap gene within AAV genome was re-packaged into viral particles and administrated onto brain slices at MOI=100K and MOI=50K in two separate rounds (i.e. different biological replicates). One week after infection, brain slices were fixed, and immunostaining was performed against GFP and Iba1+ (for microglia) on infected slices. As shown in FIG. 3, both rounds revealed an up to 6-fold increase of total microglial transduction

$$\{ \frac{( \# GFP + Iba 1 + )}{Iba 1 +} \}$$

and up to 7-fold increase of the specificity

$$\{ \frac{( \# GFP + Iba 1 + )}{\# GFP +} \}$$

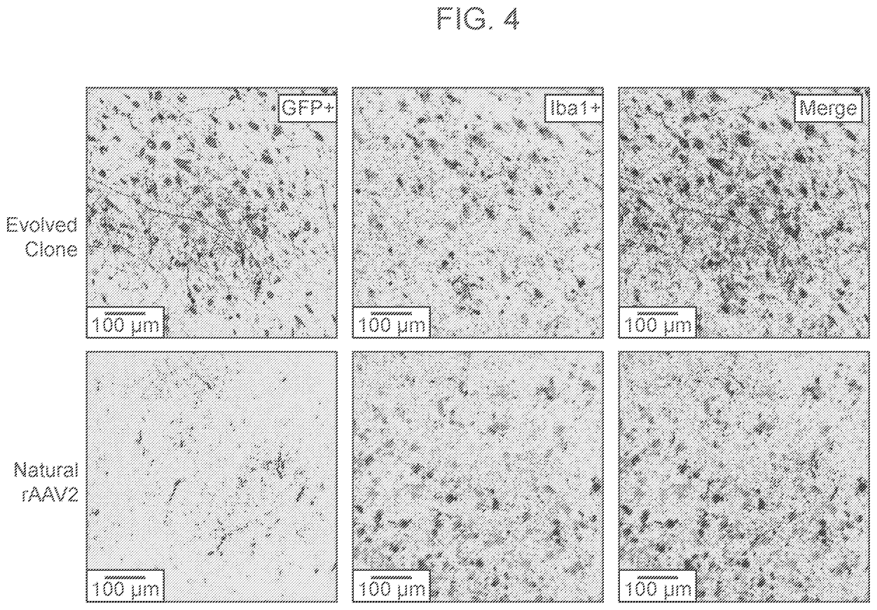

towards microglia cells. Representative images are provided in FIG. 4.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A recombinant adeno-associated virus (rAAV) virion comprising:
a1) a variant AAV capsid protein, wherein the variant AAV capsid protein comprises an amino acid sequence having at least 95% amino acid sequence identity to the amino acid sequence depicted in FIG. 6A, wherein the amino acid at position 467 is other than Gly, the amino acid at position 551 is other than Ala, the amino acid at position 665 is other than Pro, and the amino acid at position 719 is other than Glu; and
b1) a heterologous nucleic acid comprising one or more nucleotide sequences encoding one or more heterologous gene products; or
a2) a variant AAV capsid protein, wherein the variant AAV capsid protein comprises an amino acid sequence having at least 95% amino acid sequence identity to the amino acid sequence depicted in any one of FIG. 9A-9M; and
b2) a heterologous nucleic acid comprising one or more nucleotide sequences encoding one or more heterologous gene products.

2. The rAAV virion of claim 1, wherein the amino acid at position 467 is Ala, the amino acid at position 551 is Lys, the amino acid at position 665 is Ala, and the amino acid at position 719 is Asp.

3. The rAAV virion of claim 1, wherein the amino acid at position 264 is a T, Q, or A, position 448 is an S or A, position 459 is a T or N, position 470 is an S or A, position 495 is an S or T, position 533 is a D or E, position 547 is a Q, E, or T, position 555 is a T or A, position 557 is an E or D, position 561 is an M, L, or I, position 563 is an S or N, position 593 is an A, Q, or V, position 596 is an A or T, position 661 is an A, E, or T, position 662 is a V, T, or A, position 664 is a T or S, position 718 is an N or S, and position 723 is an S or T.

4. The rAAV virion of claim 1 or claim 2, wherein:
i) the amino acids at positions 264, 448, 459, 470, 495, 533, 547, 555, 557, 561, 563, 593, 596, 661, 662, 664, 718 and 723 are A, A, N, S, S, D, E, A, E, L, N, A, A, A, T, T, N, and S, respectively; or
ii) the amino acids at positions 264, 448, 459, 470, 495, 533, 547, 555, 557, 561, 563, 593, 596, 661, 662, 664, 718 and 723 are Q, S, N, A, S, E, Q, T, D, M, S, Q, T, A, V, S, S, and S, respectively; or
iii) the amino acids at positions 264, 448, 459, 470, 495, 533, 547, 555, 557, 561, 563, 593, 596, 661, 662, 664, 718 and 723 are A, A, T, S, T, D, Q, A, D, I, N, A, T, T, V, S, S, and T, respectively.

5. The rAAV virion of claim 1, wherein the amino acids at positions 264, 448, 459, 470, 495, 533, 547, 555, 557, 561, 563, 593, 596, 661, 662, 664, 718 and 723 are A, S, N, S, S, D, Q, A, E, M, S, Q, A, A, V, T, S, and S, respectively.

6. The rAAV virion of claim 1, wherein the variant capsid polypeptide comprises the amino acid sequence set forth in any one of SEQ ID NOs:53 and 81-93.

7. (canceled)

8. The rAAV virion of claim 1, wherein the rAAV virion exhibits at least 5-fold increased infectivity of a macrophage compared to the infectivity of the macrophage by a control AAV virion comprising the corresponding parental AAV capsid protein, optionally wherein the macrophage is a microglial cell.

9. The rAAV virion of claim 1, wherein the one or more heterologous gene products comprises an interfering RNA or an aptamer.

10. The rAAV virion of claim 1, wherein the wherein the one or more heterologous gene products comprises a polypeptide.

11. The rAAV virion of claim 10, wherein the polypeptide is an anti-inflammatory polypeptide, an immunosuppressive polypeptide, an immune checkpoint inhibitor, or a chimeric antigen receptor.

12. The rAAV virion of claim 10, wherein the polypeptide is a chimeric antigen receipt, a cytokine, an antibody, a T-cell receptor, an NF-κB pathway polypeptide, an interferon signaling pathway polypeptide, an immune checkpoint inhibitor, or a transcription factor.

13.-14. (canceled)

15. The rAAV virion of claim 10, wherein the polypeptide is a genome-editing enzyme.

16. The rAAV virion of claim 15, wherein the genome-editing enzyme is a CRISPR/Cas effector polypeptide, a zinc finger nuclease, or a TALEN.

17.-18. (canceled)

19. The rAAV virion of claim 1, wherein the one or more heterologous gene products comprise a CRISPR/Cas effector polypeptide and a guide RNA.

20. The rAAV virion of claim 19, wherein the guide RNA comprises a nucleotide sequence that targets a polypeptide selected from an NF-κB pathway polypeptide, an interferon signaling pathway polypeptide, apolipoprotein E (APOE), apolipoprotein C-1 (APOC1), CD22, colony stimulating factor 1 receptor (CSF1R), SPP1, tyrosine kinase binding protein (TYROBP), triggering receptor expressed on myeloid cells-2 (TREM2), valosin-containing protein (VCP), and CCR5.

21. A pharmaceutical composition comprising:
a) a recombinant adeno-associated virus virion of claim 1; and
b) a pharmaceutically acceptable excipient.

22. A method of delivering a gene product to a macrophage in an individual, the method comprising administering to the individual a recombinant adeno-associated virus (rAAV) virion according to claim 1, optionally wherein the macrophage is a microglial cell.

23. (canceled)

24. A method of treating a neurological disease or disorder in an individual, the method comprising administering to the individual in need thereof an effective amount of a recombinant adeno-associated virus (rAAV) virion according to claim 1.

25. (canceled)

26. A method of treating a cancer in an individual, the method comprising administering to an individual in need thereof an effective amount of a recombinant adeno-associated virus (rAAV) virion according to claim 1.

27.-29. (canceled)

29. An isolated nucleic acid comprising a nucleotide sequence that encodes a variant adeno-associated virus (AAV) capsid protein, wherein the variant AAV capsid protein comprises
a) an amino acid sequence having at least 95% amino acid sequence identity to the amino acid sequence set forth in SEQ ID NO:64, wherein the amino acid at position 467 is other than Gly, the amino acid at position 551 is other than Ala, the amino acid at position 665 is other than Pro, and the amino acid at position 719 is other than Glu; or
b) a variant AAV capsid protein, wherein the variant AAV capsid protein comprises an amino acid sequence having at least 95% amino acid sequence identity to the amino acid sequence set forth in any one of SEQ ID NOs: 81-95.

30. An isolated, genetically modified host cell comprising the nucleic acid of claim 29.

31. A variant adeno-associated virus (AAV) capsid protein comprising:
a) an amino acid sequence having at least 95% amino acid sequence identity to the amino acid sequence set forth in SEQ ID NO:64, wherein the amino acid at position 467 is other than Gly, the amino acid at position 551 is other than Ala, the amino acid at position 665 is other than Pro, and the amino acid at position 719 is other than Glu; or
b) a variant AAV capsid protein, wherein the variant AAV capsid protein comprises an amino acid sequence having at least 95% amino acid sequence identity to the amino acid sequence set forth in any one of SEQ ID NOs:81-95.

32. (canceled)


SEQUENCE LISTING (93 sequences)

SEQ ID NO: 1 - protein, 229 aa, Artificial sequence
  synthetic sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 2 - protein, 5 aa, Artificial sequence
  synthetic sequence
DYAMH

SEQ ID NO: 3 - protein, 17 aa, Artificial sequence
  synthetic sequence
AITWNSGHIDYADSVEG

SEQ ID NO: 4 - protein, 12 aa, Artificial sequence
  synthetic sequence
VSYLSTASSLDY

SEQ ID NO: 5 - protein, 11 aa, Artificial sequence
  synthetic sequence
RASQGIRNYLA

SEQ ID NO: 6 - protein, 7 aa, Artificial sequence
  synthetic sequence
AASTLQS

SEQ ID NO: 7 - protein, 9 aa, Artificial sequence
  synthetic sequence
QRYNRAPYT

SEQ ID NO: 8 - protein, 5 aa, Artificial sequence
  synthetic sequence
GYGMH

SEQ ID NO: 9 - protein, 17 aa, Artificial sequence
  synthetic sequence
VIWYDGSKKYYVDSVKG

SEQ ID NO: 10 - protein, 9 aa, Artificial sequence
  synthetic sequence
QMGYWHFDL

SEQ ID NO: 11 - protein, 11 aa, Artificial sequence
  synthetic sequence
RASQSVSSYLA

SEQ ID NO: 12 - protein, 7 aa, Artificial sequence
  synthetic sequence
DASNRAT

SEQ ID NO: 13 - protein, 10 aa, Artificial sequence
  synthetic sequence
QQRSNWPPLT

SEQ ID NO: 14 - protein, 5 aa, Artificial sequence
  synthetic sequence
DKTHT

SEQ ID NO: 15 - protein, 4 aa, Artificial sequence
  synthetic sequence
CPPC

SEQ ID NO: 16 - protein, 15 aa, Artificial sequence
  synthetic sequence
CPEPKSCDTPPPCPR

SEQ ID NO: 17 - protein, 12 aa, Artificial sequence
  synthetic sequence
ELKTPLGDTTHT

SEQ ID NO: 18 - protein, 10 aa, Artificial sequence
  synthetic sequence
KSCDKTHTCP

SEQ ID NO: 19 - protein, 7 aa, Artificial sequence
  synthetic sequence
KCCVDCP

SEQ ID NO: 20 - protein, 7 aa, Artificial sequence
  synthetic sequence
KYGPPCP

SEQ ID NO: 21 - protein, 15 aa, Homo sapiens
EPKSCDKTHTCPPCP

SEQ ID NO: 22 - protein, 12 aa, Homo sapiens
ERKCCVECPPCP

SEQ ID NO: 23 - protein, 17 aa, Homo sapiens
ELKTPLGDTTHTCPRCP

SEQ ID NO: 24 - protein, 12 aa, Homo sapiens
SPNMVPHAHHAQ

SEQ ID NO: 25 - protein, 45 aa, Homo sapiens
TTTPAPRPPTPAPTIASQPLSLRPEACRPAAGGAVHTRGLDFACD

SEQ ID NO: 26 - protein, 24 aa, Artificial sequence
  synthetic sequence
IYIWAPLAGTCGVLLLSLVITLYC

SEQ ID NO: 27 - protein, 23 aa, Unknown
  CD8 beta derived transmembrane domain
LGLLVAGVLVLLVSLGVAIHLCC

SEQ ID NO: 28 - protein, 25 aa, Unknown
  CD4 derived transmembrane domain
ALIVLGGVAGLLLFIGLGIFFCVRC

SEQ ID NO: 29 - protein, 23 aa, Unknown
  CD3 zeta derived transmembrane domain
LCYLLDGILFIYGVILTALFLRV

SEQ ID NO: 30 - protein, 26 aa, Unknown
  CD28 derived transmembrane domain
WVLVVVGGVLACYSLLVTVAFIIFWV

SEQ ID NO: 31 - protein, 26 aa, Unknown
  CD134 (OX40) derived transmembrane domain
VAAILGLGLVLGLLGPLAILLALYLL

SEQ ID NO: 32 - protein, 24 aa, Unknown
  CD7 derived transmembrane domain
ALPAALAVISFLLGLGLGVACVLA

SEQ ID NO: 33 - protein, 42 aa, Artificial sequence
  synthetic sequence
KRGRKKLLYIFKQPFMRPVQTTQEEDGCSCRFPEEEEGGCEL

SEQ ID NO: 34 - protein, 44 aa, Artificial sequence
  synthetic sequence
FWVRSKRSRLLHSDYMNMTPRRPGPTRKHYQPYAPPRDFAAYRS

SEQ ID NO: 35 - protein, 35 aa, Artificial sequence
  synthetic sequence
TKKKYSSSVHDPNGEYMFMRAVNTAKKSRLTDVTL

SEQ ID NO: 36 - protein, 37 aa, Artificial sequence
  synthetic sequence
RRDQRLPPDAHKPPGGGSFRTPIQEEQADAHSTLAKI

SEQ ID NO: 37 - protein, 114 aa, Artificial sequence
  synthetic sequence
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 38 - protein, 49 aa, Artificial sequence
  synthetic sequence
HQRRKYRSNKGESPVEPAEPCRYSCPREEEGSTIPIQEDYRKPEPACSP

SEQ ID NO: 39 - protein, 187 aa, Artificial sequence
  synthetic sequence
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 40 - protein, 54 aa, Artificial sequence
  synthetic sequence
HIWQLRSQCMWPRETQLLLEVPPSTEDARSCQFPEEERGERSAEEKGRLGDLWV

SEQ ID NO: 41 - protein, 60 aa, Artificial sequence
  synthetic sequence
CVKRRKPRGDVVKVIVSVQRKRQEAEGEATVIEALQAPPDVTTVAVEETIPSFTGRSPNH

SEQ ID NO: 42 - protein, 4 aa, Artificial sequence
  synthetic sequence
  SITE (2)..(3): Xaa can be any naturally occurring amino acid
  SITE (4)..(4): The amino acid at position 4 is either Leu or Ile
YXXX

SEQ ID NO: 43 - protein, 16 aa, Artificial sequence
  synthetic sequence
  SITE (2)..(3): Xaa can be any amino acid
  SITE (4)..(4): The amino acid at position 4 is either Leu or Ile
  SITE (5)..(12): Xaa can be any amino acid
  SITE (10)..(12): The amino acids at positions 10 to 12 may either be present or absent
  SITE (14)..(15): Xaa can be any naturally occurring amino acid
  SITE (16)..(16): The amino acid at position 16 is either Leu or Ile
YXXXXXXXXXXXYXXX

SEQ ID NO: 44 - protein, 447 aa, Artificial sequence
  synthetic sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 218 aa, Artificial sequence
  synthetic sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 440 aa, Artificial sequence
  synthetic sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 214 aa, Artificial sequence
  synthetic sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 448 aa, Artificial sequence
  synthetic sequence
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 215 aa, Artificial sequence
  synthetic sequence
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 448 aa, Artificial sequence
  synthetic sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, Artificial sequence
  synthetic sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 127 aa, Artificial sequence
  synthetic sequence
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVS

SEQ ID NO: 53 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGKNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 54 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 55 - protein, 735 aa, Adeno-associated virus [sequence omitted]

SEQ ID NO: 56 - protein, 736 aa, Adeno-associated virus
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 57 - protein, 736 aa, Adeno-associated virus
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 58 - protein, 734 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 59 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 60 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 61 - protein, 737 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 62 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 63 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 64 - protein, 737 aa, Adeno-associated virus
  SITE (467)..(467): Xaa can be any amino acid
  SITE (551)..(551): Xaa can be any amino acid
  SITE (665)..(665): Xaa can be any amino acid
  SITE (719)..(719): Xaa can be any amino acid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQXGPSNMSAQAKNWLPGPCYRQQRVSKTLSQNNNSNFAWTGATKYHLNGRDSLVNPGVAMATHKDDEDRFFPSSGVLIFGKQGAGXNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTXAKFASFITQYSTGQVSVEIEWELQKENSKRWNPEIQYTSNYAKSTNVDFAVNSXGVYSEPRPIGTRYLTRNL

SEQ ID NO: 65 - protein, 1368 aa, Streptococcus pyogenes [sequence omitted]

SEQ ID NO: 66 - protein, 1368 aa, Artificial sequence
  synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 67 - protein, 1368 aa, Artificial sequence
  synthetic sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 68 - protein, 1368 aa, Artificial sequence
  synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 69 - protein, 1368 aa, Artificial sequence
  synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 70 - protein, 1367 aa, Artificial sequence
  synthetic sequence
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 71 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 72 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 73 - protein, 1306 aa, Unknown
  Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 74 - protein, 1306 aa, Artificial sequence
  synthetic sequence
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 75 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 76 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 77 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 78 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 79 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 80 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 81 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSTMSAQAKNWLPGPCYRQQRVSKTLSQNNNSNFAWTGATKYHLNGRDSLVNPGVAMATHKDDEDRFFPSSGVLIFGKQGAGKNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 82 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLARTQSTGGTAGTRELLFSQAGPSNMSAQAKNWLPGPCYRQQRVSKTLSQNNNSNFAWTGATKYHLNGRDSLVNPGVAMATHKDDEDRFFPSSGVLIFGKQGAGKNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSEGVYSEPRPIGTRYLTRNL

SEQ ID NO: 83 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLARTQSTGGTAGTRELLFSQGGPSTMSAQAKNWLPGPCYRQQRVSKTLSQNNNSNFAWTGATKYHLNGRDSLVNPGVAMATHKDDEDRFFPSSGVLIFGKQGAGANNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 84 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGANNTTLENVMMTSEEEIKTTNPVATEQYGVVASNLQSSNTAPVTGTVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 85 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLARTQSTGGTAGTRELLFSQAGPSTMSAQAKNWLPGPCYRQQRVSKTLSQNNNSNFAWTGATKYHLNGRDSLVNPGVAMATHKDDEDRFFPSSGVLIFGKQGAGKNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 86 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGANNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPTVFTPAKFASFITQYSTGQVSVEIEWELQKENSKRWNPEIQYTSNYAKSTNVDFAVNNEGVYTEPRPIGTRYLTRNL

SEQ ID NO: 87 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGANNTTLENVMMTSEEEIKTTNPVATEEYGVVADNLQSSNTAPVTGAVNSQGALPGMVWQNRDVYLQGPIWAKIPHTDGNFHPSPLMGGFGLKHPPPQILIKNTPVPANPPATFSPAKFASFITQYSTGQVSVEIEWELQKENSKRWNPEIQYTSNYAKSTNVDFAVNSEGVYTEPRPIGTRYLTRNL

SEQ ID NO: 88 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGANNTTLENVMMTSEEEIKTTNPVATEEYGVVADNLQSSNTAPVTGAVNSQGALPGMVWQNRDVYLQGPIWAKIPHTDGNFHPSPLMGGFGLKHPPPQILIKNTPVPANPPAVFSPAKFASFITQYSTGQVSVEIEWELQKENSKRWNPEIQYTSNYAKSTNVDFAVNSEGVYTEPRPIGTRYLTRNL

SEQ ID NO: 89 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGKNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFSPAKFASFITQYSTGQVSVEIEWELQKENSKRWNPEIQYTSNYAKSTNVDFAVNSDGVYSEPRPIGTRYLTRNL

SEQ ID NO: 90 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGANNTALENVMMTSEEEIKTTNPVATEQYGVVASNLQSSNTAPVTGTVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 91 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGKNNTTLENVMMTSEEEIKTTNPVATEEYGVVADNLQSSNTAPVTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 92 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSAQAKNWLPGPCYRQQRVSKTLSQNNNSNFAWTGATKYHLNGRDSLVNPGVAMATHKDDEDRFFPSSGVLIFGKQGAGANNTTLENVMMTNEEEIKTTNPVATEEYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL

SEQ ID NO: 93 - protein, 737 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASSGSTNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGNRELLFSQAGPSNMSEQAKNWLPGPCYRQQRVSKTLSQNNNSNFAWTGATKYHLNGRDSLVNPGVAMATHKDDEDRFFPSSGVLIFGKQGAGKNNTALENVMMTSEEEIKTTNPVATEQYGVVASNLQSSNTAPQTGAVNSQGALPGMVWQNRDVYLQGPIWAKIPHTDGNFHPSPLMGGFGLKHPPPQILIKNTPVPANPPAVFTAAKFASFITQYSTGQVSVEIEWELQKENSKRWNPEIQYTSNYAKSTNVDFAVNSDGVYSEPRPIGTRYLTRNL